SEQUENCE LISTING (31 sequences)

SEQ ID NO: 1 - DNA, 24 bp, Artificial Sequence
  Tag
gattacaaggacgacgacgataag

SEQ ID NO: 2 - protein, 8 aa, Artificial Sequence
  synthetic
DYKDDDDK

SEQ ID NO: 3 - DNA, 60 bp, Artificial Sequence
  synthetic
atgaacgggaccgagggcccaaacttctacgtgcctttctccaacaagacgggcgtggtg

SEQ ID NO: 4 - protein, 20 aa, Artificial Sequence
  Lucy tag
MNGTEGPNFYVPFSNKTGVV

SEQ ID NO: 5 - DNA, 51 bp, Artificial Sequence
  synthetic
atgagaccccagatcctgctgctcctggccctgctgaccctaggcctggct

SEQ ID NO: 6 - protein, 17 aa, Artificial Sequence
  synthetic
MRPQILLLLALLTLGLA

SEQ ID NO: 7 - protein, 10 aa, Artificial Sequence
  motif
MAYDRYVAIC

SEQ ID NO: 8 - protein, 7 aa, Artificial Sequence
  motif-2
FSTCSSH

SEQ ID NO: 9 - protein, 8 aa, Artificial Sequence
  motif-3
PMLNPFIY

SEQ ID NO: 10 - DNA, 948 bp, homo sapiens
atggaaattgtctccacaggaaacgaaactattactgaatttgtcctccttggcttctatgacatccctgaactgcatttcttgttttttattgtattcactgctgtctatgtcttcatcatcatagggaatatgctgattattgtagcagtggttagctcccagaggctccacaaacccatgtatattttcttggcgaatctgtccttcctggatattctctacacctccgcagtgatgccaaaaatgctggagggcttcctgcaagaagcaactatctctgtggctggttgcttgctccagttctttatcttcggctctctagccacagctgaatgcttactgctggctgtcatggcatatgaccgctacctggcaatttgctacccactccactacccactcctgatggggcccagacggtacatggggctggtggtcacaacctggctctctggatttgtggtagatggactggttgtggccctggtggcccagctgaggttctgtggccccaaccacattgaccagttttactgtgactttatgcttttcgtgggcctggcttgctcggatcccagagtggctcaggtgacaactctcattctgtctgtgttctgcctcactattccttttggactgattctgacatcttatgccagaattgtggtggcagtgctgagagttcctgctggggcaagcaggagaagggctttctccacatgctcctcccacctagctgtagtgaccacattctatggaacgctcatgatcttttatgttgcaccctctgctgtccattcccagctcctctccaaggtcttctccctgctctacactgtggtcacccctctcttcaatcctgtgatctataccatgaggaacaaggaggtgcatcaggcacttcggaagattctctgtatcaaacaaactgaaacacttgattga

SEQ ID NO: 11 - protein, 315 aa, homo sapiens
MEIVSTGNETITEFVLLGFYDIPELHFLFFIVFTAVYVFIIIGNMLIIVAVVSSQRLHKPMYIFLANLSFLDILYTSAVMPKMLEGFLQEATISVAGCLLQFFIFGSLATAECLLLAVMAYDRYLAICYPLHYPLLMGPRRYMGLVVTTWLSGFVVDGLVVALVAQLRFCGPNHIDQFYCDFMLFVGLACSDPRVAQVTTLILSVFCLTIPFGLILTSYARIVVAVLRVPAGASRRRAFSTCSSHLAVVTTFYGTLMIFYVAPSAVHSQLLSKVFSLLYTVVTPLFNPVIYTMRNKEVHQALRKILCIKQTETLD

SEQ ID NO: 12 - DNA, 942 bp, homo sapiens
atgctggctagaaacaactccttagtgactgaatttattcttgctggattaacagatcatccagagttccagcaacccctctttttcctgtttctagtggtctacattgtcaccatggtaggcaaccttggcttgatcattcttttcggtctaaattctcacctccacacaccaatgtactatttcctcttcaatctctccttcattgatctctgttactcctctgttttcactcccaaaatgctaatgaactttgtatcaaaaaagaatattatctcctatgttgggtgcatgactcagctgtttttctttctcttttttgtcatctctgaatgttacatgttgacctcaatggcatatgatcgctatgtggccatctgtaatccattgctgtataaggtcaccatgtcccatcaggtctgttctatgctcacttttgctgcttacataatgggattggctggagccacggcccacaccgggtgcatgcttagactcaccttctgcagtgctaatatcatcaaccattacttgtgtgacatactccccctcctccagctttcctgcaccagcacctatgtcaacgaggtggttgttctcattgttgtgggtattaatatcatggtacccagttgtaccatcctcatttcttatgttttcattgtcactagcattcttcatatcaaatccactcaaggaagatcaaaagccttcagtacttgtagctctcatgtcattgctctgtctctgttttttgggtcagcggcattcatgtatattaaatattcttctggatctatggagcagggaaaagtttcttctgttttctacactaatgtggtgcccatgctcaatcctctcatctacagtttgaggaacaaggatgtcaaagttgcactgaggaaagctctgattaaaattcagagaagaaatatattctaa

SEQ ID NO: 13 - protein, 313 aa, homo sapiens
MLARNNSLVTEFILAGLTDHPEFQQPLFFLFLVVYIVTMVGNLGLIILFGLNSHLHTPMYYFLFNLSFIDLCYSSVFTPKMLMNFVSKKNIISYVGCMTQLFFFLFFVISECYMLTSMAYDRYVAICNPLLYKVTMSHQVCSMLTFAAYIMGLAGATAHTGCMLRLTFCSANIINHYLCDILPLLQLSCTSTYVNEVVVLIVVGINIMVPSCTILISYVFIVTSILHIKSTQGRSKAFSTCSSHVIALSLFFGSAAFMYIKYSSGSMEQGKVSSVFYTNVVPMLNPLIYSLRNKDVKVALRKALIKIQRRNIF

SEQ ID NO: 14 - DNA, 936 bp, homo sapiens
atgaaaggggcaaacctgagccaagggatggagtttgagctcttgggcctcaccactgacccccagctccagaggctgctcttcgtggtgttcctgggcatgtacacagccactctgctggggaacctggtcatgttcctcctgatccatgtgagtgccaccctgcacacacccatgtactccctcctgaagagcctctccttcttggatttctgctactcctccacggttgtgccccagaccctggtgaacttcttggccaagaggaaagtgatctcttattttggctgcatgactcagatgttcttctatgcgggttttgccaccagtgagtgctatctcatcgctgccatggcctatgaccgctatgccgctatttgtaaccccctgctctactcaaccatcatgtctcctgaggtctgtgcctcgctgattgtgggctcctacagtgcaggattcctcaattctcttatccacactggctgtatctttagtctgaaattctgcggtgctcatgtcgtcactcacttcttctgtgatgggccacccatcctgtccttgtcttgtgtagacacctcactgtgtgagatcctgctcttcatttttgctggtttcaaccttttgagctgcaccctcaccatcttgatctcctacttcttaattctcaacaccatcctgaaaatgagctcggcccagggcaggtttaaggcattttccacctgtgcatcccacctcactgccatctgcctcttctttggcacaacactttttatgtacctgcgccccaggtccagctactccttgacccaggaccgcacagttgctgtcatctacacagtggtgatcccagtgctgaaccccctcatgtactctttgagaaacaaggatgtgaagaaagctttaataaaggtttggggtaggaaaacaatggaatga

SEQ ID NO: 15 - protein, 311 aa, homo sapiens
MKGANLSQGMEFELLGLTTDPQLQRLLFVVFLGMYTATLLGNLVMFLLIHVSATLHTPMYSLLKSLSFLDFCYSSTVVPQTLVNFLAKRKVISYFGCMTQMFFYAGFATSECYLIAAMAYDRYAAICNPLLYSTIMSPEVCASLIVGSYSAGFLNSLIHTGCIFSLKFCGAHVVTHFFCDGPPILSLSCVDTSLCEILLFIFAGFNLLSCTLTILISYFLILNTILKMSSAQGRFKAFSTCASHLTAICLFFGTTLFMYLRPRSSYSLTQDRTVAVIYTVVIPVLNPLMYSLRNKDVKKALIKVWGRKTME

SEQ ID NO: 16 - DNA, 927 bp, homo sapiens
atgaccatggaaaattattctatggcagctcagtttgtcttagatggtttaacacagcaagcagagctccagctgcccctcttcctcctgttcctgggaatctatgtggtcacagtagtgggcaacctgggcatgattctcctgattgcagtcagccctctacttcacacccccatgtactatttcctcagcagcttgtccttcgtcgatttctgctattcctctgtcattactcccaaaatgctggtgaacttcctaggaaagaagaatacaatcctttactctgagtgcatggtccagctctttttctttgtggtctttgtggtggctgagggttacctcctgactgccatggcatatgatcgctatgttgccatctgtagcccactgctttataatgcgatcatgtcctcatgggtctgctcactgctagtgctggctgccttcttcttgggctttctctctgccttgactcatacaagtgccatgatgaaactgtccttttgcaaatcccacattatcaaccattacttctgtgatgttcttcccctcctcaatctctcctgctccaacacacacctcaatgagcttctactttttatcattgcggggtttaacaccttggtgcccaccctagctgttgctgtctcctatgccttcatcctctacagcatccttcacatccgctcctcagagggccggtccaaagcttttggaacatgcagctctcatctcatggctgtggtgatcttctttgggtccattaccttcatgtatttcaagcccccttcaagtaactccctggaccaggagaaggtgtcctctgtgttctacaccacggtgatccccatgctgaaccctttaatatacagtctgaggaataaggatgtgaagaaagcattaaggaaggtcttagtaggaaaatga

SEQ ID NO: 17 - protein, 308 aa, homo sapiens
MTMENYSMAAQFVLDGLTQQAELQLPLFLLFLGIYVVTVVGNLGMILLIAVSPLLHTPMYYFLSSLSFVDFCYSSVITPKMLVNFLGKKNTILYSECMVQLFFFVVFVVAEGYLLTAMAYDRYVAICSPLLYNAIMSSWVCSLLVLAAFFLGFLSALTHTSAMMKLSFCKSHIINHYFCDVLPLLNLSCSNTHLNELLLFIIAGFNTLVPTLAVAVSYAFILYSILHIRSSEGRSKAFGTCSSHLMAVVIFFGSITFMYFKPPSSNSLDQEKVSSVFYTTVIPMLNPLIYSLRNKDVKKALRKVLVGK

SEQ ID NO: 18 - DNA, 936 bp, homo sapiens
atgactgggggaggaaatattacagaaatcacctatttcatcctgctgggattctcagattttcccaggatcataaaagtgctcttcactatattcctggtgatctacattacatctctggcctggaacctctccctcattgttttaataaggatggattcccacctccatacacccatgtatttcttcctcagtaacctgtccttcatagatgtctgctatatcagctccacagtccccaagatgctctccaacctcttacaggaacagcaaactatcacttttgttggttgtattattcagtactttatcttttcaacgatgggactgagtgagtcttgtctcatgacagccatggcttatgatcgttatgctgccatttgtaaccccctgctctattcatccatcatgtcacccaccctctgtgtttggatggtactgggagcctacatgactggcctcactgcttctttattccaaattggtgctttgcttcaactccacttctgtgggtctaatgtcatcagacatttcttctgtgacatgccccaactgttaatcttgtcctgtactgacactttctttgtacaggtcatgactgctatattaaccatgttctttgggatagcaagtgccctagttatcatgatatcctatggctatattggcatctccatcatgaagatcacttcagctaaaggcaggtccaaggcattcaacacctgtgcttctcatctaacagctgtttccctcttctatacatcaggaatctttgtctatttgagttccagctctggaggttcttcaagctttgacagatttgcatctgttttctacactgtggtcattcccatgttaaatcccttgatttacagtttgaggaacaaagaaattaaagatgccttaaagaggttgcaaaagagaaagtgctgctga

SEQ ID NO: 19 - protein, 311 aa, homo sapiens
MTGGGNITEITYFILLGFSDFPRIIKVLFTIFLVIYITSLAWNLSLIVLIRMDSHLHTPMYFFLSNLSFIDVCYISSTVPKMLSNLLQEQQTITFVGCIIQYFIFSTMGLSESCLMTAMAYDRYAAICNPLLYSSIMSPTLCVWMVLGAYMTGLTASLFQIGALLQLHFCGSNVIRHFFCDMPQLLILSCTDTFFVQVMTAILTMFFGIASALVIMISYGYIGISIMKITSAKGRSKAFNTCASHLTAVSLFYTSGIFVYLSSSSGGSSSFDRFASVFYTVVIPMLNPLIYSLRNKEIKDALKRLQKRKCC

SEQ ID NO: 20 - DNA, 951 bp, homo sapiens
atgggaaaaccaggcagagtgaaccaaaccactgtttcagacttcctccttttaggactctctgagcggccagaggagcagcctcttctgtttggcatcttccttggcatgtacctggtcaccatggtggggaacctgctcattatcctggccatcagctctgacccacacctccatactcccatgtacttctttctggccaacctgtcattaactgatgcctgtttcacttctgcctccatccccaaaatgctggccaacattcatacccagagtcagatcatctcctattctgggtgtcttgcacagctatatttcctccttatgtttggtggccttgacaactgcctgctggctgtgatggcatatgaccgctatgtggccatctgccaaccactccattacagcacatctatgagtccccagctctgtgcactaatgctgggtgtgtgctgggtgctaaccaactgtcctgccctgatgcacacactgttgctgacccgcgtggctttctgtgcccagaaagccatccctcatttctactgtgatcccagtgctctcctgaagcttgcctgctcagatacccatgtaaacgagctgatgatcatcaccatgggcttgctgttcctcactgttcccctcctgctgatcgtcttctcctatgtccgcattttctgggctgtgtttggcatctcatctcctggagggagatggaaggccttctctacctgtggttctcatctcacggtggttctgctcttctatgggtctcttatgggtgtgtatttacttcctccatcaacttactctacagagagggaaagtagggctgctgttctctatatggtgattattcccatgctaaacccattcatttatagcttgaggaacagagacatgaaggaggctttgggtaaactttttgtcagtggaaaaacattctttttatga

SEQ ID NO: 21 - protein, 316 aa, homo sapiens
MGKPGRVNQTTVSDFLLLGLSERPEEQPLLFGIFLGMYLVTMVGNLLIILAISSDPHLHTPMYFFLANLSLTDACFTSASIPKMLANIHTQSQIISYSGCLAQLYFLLMFGGLDNCLLAVMAYDRYVAICQPLHYSTSMSPQLCALMLGVCWVLTNCPALMHTLLLTRVAFCAQKAIPHFYCDPSALLKLACSDTHVNELMIITMGLLFLTVPLLLIVFSYVRIFWAVFGISSPGGRWKAFSTCGSHLTVVLLFYGSLMGVYLLPPSTYSTERESRAAVLYMVIIPMLNPFIYSLRNRDMKEALGKLFVSGKTFFL

SEQ ID NO: 22 - DNA, 948 bp, homo sapiens
atgtccataaccaaagcctggaacagctcatcagtgaccatgttcatcctcctgggattcacagaccatccagaactccaggccctcctctttgtgaccttcctgggcatctatcttaccaccctggcctggaacctggccctcatttttctgatcagaggtgacacccatctgcacacacccatgtacttcttcctaagcaacttatctttcattgacatctgctactcttctgctgtggctcccaatatgctcactgacttcttctgggagcagaagaccatatcatttgtgggctgtgctgctcagttttttttctttgtcggcatgggtctgtctgagtgcctcctcctgactgctatggcatacgaccgatatgcagccatctccagcccccttctctaccccactatcatgacccagggcctctgtacacgcatggtggttggggcatatgttggtggcttcctgagctccctgatccaggccagctccatatttaggcttcacttttgcggacccaacatcatcaaccacttcttctgcgacctcccaccagtcctggctctgtcttgctctgacaccttcctcagtcaagtggtgaatttcctcgtggtggtcactgtcggaggaacatcgttcctccaactccttatctcctatggttacatagtgtctgcggtcctgaagatcccttcagcagagggccgatggaaagcctgcaacacgtgtgcctcgcatctgatggtggtgactctgctgtttgggacagcccttttcgtgtacttgcgacccagctccagctacttgctaggcagggacaaggtggtgtctgttttctattcattggtgatccccatgctgaaccctctcatttacagtttgaggaacaaagagatcaaggatgccctgtggaaggtgttggaaaggaagaaagtgttttcttag

SEQ ID NO: 23 - protein, 315 aa, homo sapiens
MSITKAWNSSSVTMFILLGFTDHPELQALLFVTFLGIYLTTLAWNLALIFLIRGDTHLHTPMYFFLSNLSFIDICYSSAVAPNMLTDFFWEQKTISFVGCAAQFFFFVGMGLSECLLLTAMAYDRYAAISSPLLYPTIMTQGLCTRMVVGAYVGGFLSSLIQASSIFRLHFCGPNIINHFFCDLPPVLALSCSDTFLSQVVNFLVVVTVGGTSFLQLLISYGYIVSAVLKIPSAEGRWKACNTCASHLMVVTLLFGTALFVYLRPSSSYLLGRDKVVSVFYSLVIPMLNPLIYSLRNKEIKDALWKVLERKKVFS

SEQ ID NO: 24 - DNA, 930 bp, homo sapiens
atggaaccagagaatgacacagggatttcagaatttgttcttctgggactttctgaggaaccagaattgcagcccttcctctttgggctgtttctgtccatgtacctggtcactgtgctcgggaatctgctcatcatcctggccacaatctcagactcccacctccacacccccatgtacttcttcctctccaacctgtcctttgcagacatctgtttcatctccactacaatcccaaagatgctcattaacatccagacacagagcagagtcatcacctatgcaggctgcatcacccagatgtgcttttttgtactttttggagggttagacagcttactcctggctgtgatggcctatgatcggtttgtggccatctgtcatcctctgcactacacagtcatcatgaaccctcggctctgtggactcctggttctggcatcctggatgattgctgccctgaattccttgtcacaaagcttaatggtattgtggctgtccttctgcacagacttggaaatcccccactttttctgtgaacttaatcaggtcatccaccttgcctgttctgacacctttcttaatgacatggggatgtattttgcagcagggctgctggctggtggtccccttgtggggatcctttgctcttactctaagatagtttcctccatacgtgcaatctcatcagctcaggggaagtacaaggcattttccacctgtgcatcacacctctcagttgtctctttattttgttgtacgggcctaggtgtgtaccttacttctgctgcaacccacaactcacacacaagtgcaacagcctcagtgatgtacactgtggccacccccatgctgaacccctttatctacagtctgaggaataaagacataaagagggctctgaaaatgtccttcagaggaaagcaataa

SEQ ID NO: 25 - protein, 309 aa, homo sapiens
MEPENDTGISEFVLLGLSEEPELQPFLFGLFLSMYLVTVLGNLLIILATISDSHLHTPMYFFLSNLSFADICFISTTIPKMLINIQTQSRVITYAGCITQMCFFVLFGGLDSLLLAVMAYDRFVAICHPLHYTVIMNPRLCGLLVLASWMIAALNSLSQSLMVLWLSFCTDLEIPHFFCELNQVIHLACSDTFLNDMGMYFAAGLLAGGPLVGILCSYSKIVSSIRAISSAQGKYKAFSTCASHLSVVSLFCCTGLGVYLTSAATHNSHTSATASVMYTVATPMLNPFIYSLRNKDIKRALKMSFRGKQ

SEQ ID NO: 26 - DNA, 930 bp, homo sapiens
atgaagagaaagaacttcacagaagtgtcagaattcattttcttgggattttctagctttggaaagcatcagataaccctctttgtggttttcctaactgtctacattttaactctggttgctaacatcatcattgtgactatcatctgcattgaccatcatctccacactcccatgtatttcttcctaagcatgctggctagttcagagacggtgtacacactggtcattgtgccacgaatgcttttgagcctcatttttcataaccaacctatctccttggcaggctgtgctacacaaatgttcttttttgttatcttggccactaataattgcttcctgcttactgcaatggggtatgaccgctatgtggccatctgcagacccctgagatacactgtcatcatgagcaagggactatgtgcccagctggtgtgtgggtcctttggcattggtctgactatggcagttctccatgtgacagccatgttcaatttgccgttctgtggcacagtggtagaccacttcttttgtgacatttacccagtcatgaaactttcttgcattgataccactatcaatgagataataaattatggtgtaagttcatttgtgatttttgtgcccataggcctgatatttatctcctatgtccttgtcatctcttccatccttcaaattgcctcagctgagggccggaagaagacctttgccacctgtgtctcccacctcactgtggttattgtccactgtggctgtgcctccattgcctacctcaagccgaagtcagaaagttcaatagaaaaagaccttgttctctcagtgacgtacaccatcatcactcccttgctgaaccctgttgtttacagtctgagaaacaaggaggtaaaggatgccctatgcagagttgtgggcagaaatatttcttaa

SEQ ID NO: 27 - protein, 309 aa, homo sapiens
MKRKNFTEVSEFIFLGFSSFGKHQITLFVVFLTVYILTLVANIIIVTIICIDHHLHTPMYFFLSMLASSETVYTLVIVPRMLLSLIFHNQPISLAGCATQMFFFVILATNNCFLLTAMGYDRYVAICRPLRYTVIMSKGLCAQLVCGSFGIGLTMAVLHVTAMFNLPFCGTVVDHFFCDIYPVMKLSCIDTTINEIINYGVSSFVIFVPIGLIFISYVLVISSILQIASAEGRKKTFATCVSHLTVVIVHCGCASIAYLKPKSESSIEKDLVLSVTYTIITPLLNPVVYSLRNKEVKDALCRVVGRNIS

SEQ ID NO: 28 - DNA, 945 bp, homo sapiens
atggaaaaaagaaatctaacagttgtcagggaattcgtccttctgggacttcctagctcagcagagcagcagcacctcctgtctgtgctctttctctgtatgtatttagccaccaccttggggaacatgctcatcattgcgacgattggctttgactctcacctccattcccctatgtacttcttccttagtaacttggcctttgttgacatctgctttacgtcgactacagtcccccaaatggtagtgaatatcttgactggcaccaagactatctcttttgcaggctgcctcacccagctcttcttcttcgtttcttttgtgaatatggacagcctccttctgtgtgtgatggcgtatgatagatatgtggcgatttgccaccccttacattacaccgccagaatgaacctgtgcctttgtgtccagctagtggctggactgtggcttgttacttacctccacgccctcctgcatactgtcctaatagcacagctgtccttctgtgcctccaatatcatccatcatttcttctgtgatctcaatcctctcctgcagctctcttgctctgacgtctccttcaatgtaatgatcatttttgcagtaggaggtctattggctctcacgccccttgtctgtatcctcgtatcttatggacttatcttctccactgttctgaagatcacctctactcagggcaagcagagagctgtttccacctgcagctgccacctgtcagtggtggtgttgttttacggcacagccatcgccgtctatttcagcccttcatccccccatatgcctgagagcgacactctgtcaaccatcatgtattcaatggtggctccgatgctgaatcctttcatctataccctaaggaacagggatatgaagaggggacttcagaaaatgcttctcaagtgcacagtctttcagcagcaataa

SEQ ID NO: 29 - protein, 314 aa, homo sapiens
MEKRNLTVVREFVLLGLPSSAEQQHLLSVLFLCMYLATTLGNMLIIATIGFDSHLHSPMYFFLSNLAFVDICFTSTTVPQMVVNILTGTKTISFAGCLTQLFFFVSFVNMDSLLLCVMAYDRYVAICHPLHYTARMNLCLCVQLVAGLWLVTYLHALLHTVLIAQLSFCASNIIHHFFCDLNPLLQLSCSDVSFNVMIIFAVGGLLALTPLVCILVSYGLIFSTVLKITSTQGKQRAVSTCSCHLSVVVLFYGTAIAVYFSPSSPHMPESDTLSTIMYSMVAPMLNPFIYTLRNRDMKRGLQKMLLKCTVFQQQ

SEQ ID NO: 30 - DNA, 945 bp, homo sapiens
atggagaacaacacagaggtgactgaattcatccttgtggggttaactgatgacccagaactgcagatcccactcttcatagtcttccttttcatctacctcatcactctggttgggaacctggggatgattgaattgattctactggactcctgtctccacacccccatgtacttcttcctcagtaacctctccctggtggactttggttattcctcagctgtcactcccaaggtgatggtggggtttctcacaggagacaaattcatattatataatgcttgtgccacacaattcttcttctttgtagcctttatcactgcagaaagtttcctcctggcatcaatggcctatgaccgctatgcagcattgtgtaaacccctgcattacaccaccaccatgacaacaaatgtatgtgcttgcctggccataggctcctacatctgtggtttcctgaatgcatccattcatactgggaacactttcaggctctccttctgtagatccaatgtagttgaacactttttctgtgatgctcctcctctcttgactctctcatgttcagacaactacatcagtgagatggttattttttttgtggtgggattcaatgacctcttttctatcctggtaatcttgatctcctacttatttatatttatcaccatcatgaagatgcgctcacctgaaggacgccagaaggccttttctacttgtgcttcccaccttactgcagtttccatcttttatgggacaggaatctttatgtacttacgacctaactccagccatttcatgggcacagacaaaatggcatctgtgttctatgccatagtcattcccatgttgaatccactggtctacagcctgaggaacaaagaggttaagagtgcctttaaaaagactgtagggaaggcaaaggcctctataggattcatattttaa

SEQ ID NO: 31 - protein, 314 aa, homo sapiens
MENNTEVTEFILVGLTDDPELQIPLFIVFLFIYLITLVGNLGMIELILLDSCLHTPMYFFLSNLSLVDFGYSSAVTPKVMVGFLTGDKFILYNACATQFFFFVAFITAESFLLASMAYDRYAALCKPLHYTTTMTTNVCACLAIGSYICGFLNASIHTGNTFRLSFCRSNVVEHFFCDAPPLLTLSCSDNYISEMVIFFVVGFNDLFSILVILISYLFIFITIMKMRSPEGRQKAFSTCASHLTAVSIFYGTGIFMYLRPNSSHFMGTDKMASVFYAIVIPMLNPLVYSLRNKEVKSAFKKTVGKAKASIGFIF

(19) United States
(12) Patent Application Publication
(10) Pub. No.: US 2023/0065799 A1
(43) Pub. Date: Mar. 2, 2023

(54) METHOD FOR ATTRIBUTING OLFACTORY TONALITIES TO OLFACTORY RECEPTOR ACTIVATION AND METHODS FOR IDENTIFYING COMPOUNDS HAVING THE ATTRIBUTED TONALITIES

(71) Applicant: FIRMENICH SA, Satigny (CH)

(72) Inventors: Ben SMITH, Plainsboro, NJ (US); Patrick PFISTER, Plainsboro, NJ (US); Lily WU, Plainsboro, NJ (US); Hyo Young JEONG, Plainsboro, NJ (US); Daniel RAPS, Plainsboro, NJ (US)

(21) Appl. No.: 17/635,651

(22) PCT Filed: Oct. 2, 2020
(86) PCT No.: PCT/EP2020/077711

§ 371 Date: Feb. 15, 2022

Related U.S. Application Data
(60) Provisional application No. 62/911,096, filed on Oct. 4, 2019.

(30) Foreign Application Priority Data
Nov. 28, 2019 (EP) 19212031.9

(51) Int. Cl. G16C 20/30; C11B 9/00; G16C 60/00

(52) U.S. Cl. CPC G16C 20/30; C11B 9/00; G16C 60/00

(57) ABSTRACT

The present invention relates to the perfumery industry. More particularly, the present invention relates to assays and methods for screening and identifying compositions and/or ingredients that intensify a subjects perception of target odorant compounds based on the use of particular olfactory receptors activated by the target odorant compound.

### CROSS-REFERENCE TO RELATED APPLICATIONS

This application is a U.S. National Phase Application of International Patent Application No. PCT/EP2020/077711, filed Oct. 2, 2020, which claims priority to U.S. Provisional Application No. 62/911,096, filed Oct. 4, 2019, and European Patent Application No. 19212031.9, filed Nov. 28, 2019, each of which is hereby incorporated by reference herein.

### REFERENCE TO AN ELECTRONIC SEQUENCE LISTING

This application contains an electronic sequence listing. The contents of the electronic sequence listing (36803-337_Imported_ST25.txt; Size: 46,643 bytes; and Date of Creation: Jul. 26, 2022) is herein incorporated by reference in its entirety.

### FIELD OF THE INVENTION

The present invention is directed to the field of odorant and aroma receptors and tonality assays that can be used to identify olfactory receptors and compounds having at least one tonality.

### BACKGROUND

Following the initial identification of odorant receptors in 1991, much has been done to advance the understanding of olfaction and how humans perceive volatile chemicals. At the periphery of the human olfactory system, the main olfactory epithelium lining the nasal cavity harbors several million olfactory sensory neurons (OSNs), each expressing a single member from a family of approximately 400 intact odorant (olfactory) receptor (OR) genes. Each OR can be activated by several volatile compounds while, in turn, a single volatile compound can also activate several ORs. This results in a combinatorial OSN activation code for each volatile chemical, and mixture thereof. Each receptor is stochastically expressed in OSNs in a monogenic and monoallelic fashion, yielding one OSN type for each OR allele present in the genome. Depending on the frequency of gene choice for each OR, the subset of the total OSN population dedicated to each OSN type varies. The OSN-type subpopulations each provide a discrete channel of information about the molecular identity, or identities, and concentrations of volatile compounds with which they are in contact at any given time. The level of activity induced in each OSN of each OSN type varies according to the concentration of volatile compounds to which its OR is receptive, as well as according to the binding and activation parameters associated with each OR-compound pair. Further, some OR-ligand pairs induce or enhance OSN activity, while others reduce or prevent OSN activity. This can occur competitively, where compounds compete to bind an OR, or non-competitively, where more than one compound can bind an OR simultaneously. The resulting combinatorial logic of these approximately 400 input channels (i.e. the peripheral olfactory system) is transformed by subsequent, downstream, neurological networks and results in our sense of smell.

The fragrance and flavor (F&F) industry is constantly in search for new ingredients, novel perfumery and flavor applications, improved sensory experiences, and compounds that are more stable, biodegradable and non-toxic.

Because an odorant or aroma molecule may interact with several olfactory receptors (ORs), it is often difficult to infer the tonality of volatile compounds based on its chemical structure alone.

Conversely, because each OR may interact with several odorant and aroma molecules possessing different chemical structures and evoke different sets of tonalities, it has often been difficult to infer the nature of the information encoded by an OR.

There remains a need to predict the olfactory tonality of a given compound or composition based on OR activation and to facilitate new perfumery and flavor ingredient discovery relevant to the F&F industry.

### SUMMARY OF THE INVENTION

In one aspect, a method is provided for associating an at least one olfactory tonality to an olfactory receptor comprising:
(a) providing an olfactory receptor;
(b) contacting the olfactory receptor and a compound having at least one known tonality;
(c) determining whether the compound activates the olfactory receptor;
(d) repeating steps (b) and (c) with a compound having at least one known tonality, wherein the compound of step (d) is different than the compound of the preceding iterations of steps (b) and (c);
(e) categorizing as a subset, the compounds from steps (b) through (d) that activate the olfactory receptor;
(f) identifying an at least one tonality common to the subset of compounds; and
(g) assigning the identified at least one tonality to the olfactory receptor.

In one aspect, a method is provided for screening at least one compound having a particular tonality comprising:
(a) providing an olfactory receptor having the identified at least one tonality;
(b) contacting the olfactory receptor with an at least one compound;
(c) determining whether the at least one compound activates the olfactory receptor;
(d) associating the at least one compound to the identified at least one tonality if the at least one compound activates the olfactory receptor.

In some aspects, a plurality of olfactory receptors, each having a different identified at least one tonality, is provided in step (a) and a plurality of identified tonalities is associated in step (d) to the compound or combination of compounds that activate the plurality of olfactory receptors according to steps (b) and (c) in the aspect described above.

In one aspect, a method is provided for screening at least one compound for an earthy tonality comprising:
(a) providing a polypeptide comprising an amino acid sequence having at least 90% sequence identity to SEQ ID NO:11;
(b) contacting the polypeptide with the at least one compound;
(c) determining whether the at least one compound activates the polypeptide; and
(d) associating an earthy tonality to the at least one compound if the at least one compound activates the polypeptide;
wherein the polypeptide is an olfactory receptor.

In one aspect, a method is provided for screening at least one compound for a coumarinic tonality comprising:
(a) providing a polypeptide comprising an amino acid sequence having at least 90% sequence identity to SEQ ID NO:13;
(b) contacting the polypeptide with the at least one compound;
(c) determining whether the at least one compound activates the polypeptide; and
(d) associating a coumarinic tonality to the at least one compound if the at least one compound activates the polypeptide;
wherein the polypeptide is an olfactory receptor.

In one aspect, a method is provided for screening at least one compound for an lactonic coconut tonality comprising:
(a) providing a polypeptide comprising an amino acid sequence having at least 90% sequence identity to SEQ ID NO:15;
(b) contacting the polypeptide with the at least one compound;
(c) determining whether the at least one compound activates the polypeptide; and
(d) associating a lactonic coconut tonality to the at least one compound if the at least one compound activates the polypeptide;
wherein the polypeptide is an olfactory receptor.

In one aspect, a method is provided for screening at least one compound for a fenugreek tonality comprising:
(a) providing a polypeptide comprising an amino acid sequence having at least 90% sequence identity to SEQ ID NO:17;
(b) contacting the polypeptide with the at least one compound;
(c) determining whether the at least one compound activates the polypeptide; and
(d) associating a fenugreek tonality to the at least one compound if the at least one compound activates the polypeptide;
wherein the polypeptide is an olfactory receptor.

In one aspect, a method is provided for screening at least one compound for a powdery musk tonality comprising:
(a) providing a polypeptide comprising an amino acid sequence having at least 90% sequence identity to SEQ ID NO:19;
(b) contacting the polypeptide with the at least one compound;
(c) determining whether the at least one compound activates the polypeptide; and
(d) associating a powdery musk tonality to the at least one compound if the at least one compound activates the polypeptide;
wherein the polypeptide is an olfactory receptor.

In one aspect, a method is provided for screening at least one compound for an animalic musk tonality comprising:
(a) providing a polypeptide comprising an amino acid sequence having at least 90% sequence identity to SEQ ID NO:21;
(b) contacting the polypeptide with the at least one compound;
(c) determining whether the at least one compound activates the polypeptide; and
(d) associating an animalic musk tonality to the at least one compound if the at least one compound activates the polypeptide;
wherein the polypeptide is an olfactory receptor.

In one aspect, a method is provided for screening at least one compound for a violet tonality comprising:
(a) providing a polypeptide comprising an amino acid sequence having at least 90% sequence identity to SEQ ID NO:23;
(b) contacting the polypeptide with the at least one compound;
(c) determining whether the at least one compound activates the polypeptide; and
(d) associating a violet tonality to the at least one compound if the at least one compound activates the polypeptide;
wherein the polypeptide is an olfactory receptor.

In one aspect, a method is provided for screening at least one compound for a blonde wood tonality comprising:
(a) providing a polypeptide comprising an amino acid sequence having at least 90% sequence identity to SEQ ID NO:25;
(b) contacting the polypeptide with the at least one compound;
(c) determining whether the at least one compound activates the polypeptide; and
(d) associating a blonde wood tonality to the at least one compound if the at least one compound activates the polypeptide;
wherein the polypeptide is an olfactory receptor.

In one aspect, a method is provided for screening at least one compound for a muguet tonality comprising:
(a) providing a polypeptide comprising an amino acid sequence having at least 90% sequence identity to SEQ ID NO:27;
(b) contacting the polypeptide with the at least one compound;
(c) determining whether the at least one compound activates the polypeptide; and
(d) associating a muguet tonality to the at least one compound if the at least one compound activates the polypeptide;
wherein the polypeptide is an olfactory receptor.

In one aspect, a method is provided for screening at least one compound for a linalic tonality comprising:
(a) providing a polypeptide comprising an amino acid sequence having at least 90% sequence identity to SEQ ID NO:29;
(b) contacting the polypeptide with the at least one compound;
(c) determining whether the compound or combination of compounds activates the polypeptide; and
(d) associating a linalic tonality to the at least one compound if the at least one compound activates the polypeptide;
wherein the polypeptide is an olfactory receptor.

In one aspect, a method is provided for screening at least one compound for a jasmine tonality comprising:
(a) providing a polypeptide comprising an amino acid sequence having at least 90% sequence identity to SEQ ID NO:31;
(b) contacting the polypeptide with the at least one compound;
(c) determining whether the compound or combination of compounds activates the polypeptide; and
(d) associating a jasmine tonality to the at least one compound if the at least one compound activates the polypeptide;
wherein the polypeptide is an olfactory receptor.

In some aspects, the present disclosure provides at least one compound identified by the methods according to certain aspects described herein.

In one aspect, a method is provided for replacing a compound in a composition having a fragrance comprising:
(a) selecting a compound in a composition having a fragrance;
(b) identifying a tonality of the compound in the composition;
(c) providing an olfactory receptor associated with the tonality;
(d) contacting the olfactory receptor and a test compound;
(e) determining whether the test compound activates the olfactory receptor; and
(f) replacing the selected compound in the composition with the test compound if the test compound activates the olfactory receptor.

In one aspect, a method is provided for replacing at least one compound in a plurality of compounds in a composition having a fragrance comprising:
(a) selecting a plurality of compounds in a composition having a fragrance, wherein each compound in the plurality has at least one tonality;
(b) identifying the tonalities of the plurality of compounds;
(c) providing a plurality of olfactory receptors, wherein greater than one of the olfactory receptors was assigned the same tonality as at least one of the compounds in the plurality of compounds;
(d) contacting at least one test compound with greater than one of the olfactory receptors;
(e) determining whether the at least one test compound activates greater than one of the olfactory receptors; and
(f) replacing greater than one of the compounds in the plurality of compounds with the test compound if the test compound activates the same olfactory receptors as the greater than one of the compounds in the plurality of compounds.

In one aspect, a method is provided for producing a composition having a desired fragrance comprising:
(a) choosing a desired fragrance;
(b) determining a plurality of tonalities that, in combination, will produce the desired fragrance; and
(c) adding to the composition at least one compound that has the plurality of tonalities.

In some aspects, the at least one compound having the plurality of tonalities is identified according to the methods according to certain aspects described herein.

In one aspect, a method is provided for removing at least one compound in a composition having a desired fragrance comprising:
(a) choosing a composition having a desired fragrance, wherein the fragrance is comprised of at least one tonality;
(b) identifying at least one olfactory receptor associated with the at least one tonality;
(c) selecting at least one compound in the composition having the desired fragrance;
(d) contacting the at least one compound and the at least one olfactory receptor;
(e) determining whether the compound inhibits the at least one olfactory receptor; and
(f) removing the at least one compound from the composition having a desired fragrance if the at least one compound inhibits the at least one olfactory receptor.

In one aspect, a method is provided for adding at least one compound to a composition having a fragrance comprising:
(a) choosing a composition having a fragrance, wherein the fragrance is comprised of at least one tonality;
(b) identifying at least one olfactory receptor associated with the at least one tonality;
(c) contacting the at least one olfactory receptor and at least one test compound;
(d) determining whether the at least one test compound inhibits the at least one olfactory receptor; and
(e) adding the at least one test compound to the composition having a fragrance if the at least one test compound inhibits the at least one olfactory receptor;
wherein the at least one olfactory receptor was assigned a tonality that is not desired in the fragrance.

In one aspect, a method is provided for adding at least one compound to a composition having a fragrance comprising:
(a) choosing a composition having a fragrance, wherein the fragrance is comprised of at least one tonality;
(b) identifying a first olfactory receptor associated with a first of the at least one tonality, wherein the first tonality is desired in the fragrance;
(c) contacting the first olfactory receptor and a test compound;
(d) determining whether the test compound activates the first olfactory receptor;
(e) identifying a second olfactory receptor associated with a second of the at least one tonality, wherein the second tonality is not desired in the fragrance;
(f) contacting the second olfactory receptor and the test compound;
(g) determining whether the test compound inhibits the second olfactory receptor;
and
(h) adding the test compound to the composition having a fragrance if the test compound activates the first olfactory receptor and inhibits the second olfactory receptor.

In some aspects, the added test compound replaces a compound in the fragrance.

### BRIEF DESCRIPTION OF THE FIGURES

FIG. 1 summarizes how specific volatile compounds acquire at least one olfactory tonality through an OR activation. Compounds activating multiple receptors exhibit the corresponding tonalities associated with the activated ORs.

FIG. 2 shows the correlation between OR activation and tonality specifically around receptors OR5AU1 (associated with a coconut tonality), OR8B3 (associated with a coumarinic tonality) and OR8D1 (associated with a fenugreek tonality).

FIG. 3 shows the human odorant receptor OR11A1 activation ranking obtained from a single-concentration high-throughput screening process using a large and diverse set of volatile compounds.

FIG. 4 shows the results of human odorant receptor OR11A1 dose-response experiments obtained for four active agonists (Geosmin, Vulcanolide, Fenchyl Alcohol and Patchouli Alcohol) capturing both potency and efficacy of each compound for the receptor.

FIG. 5 shows the molecular receptive range OR11A1 according to potency and efficacy obtained from dose-response experiments with hits from FIG. 3. The most active agonists are highlighted.

FIG. 6 shows the chemical structures of corresponding agonists of the four most active agonists and a partial agonist of OR11A1.

FIG. 7 shows the molecular receptive range of OR8B3 agonists according to potency and efficacy, and highlights the most active agonists, wherein a coumarinic tonality is common.

FIG. 8 shows the molecular receptive range of OR5AU1 agonists according to potency and efficacy, and highlights the most active agonists, wherein a lactonic coconut tonality is common.

FIG. 9 shows the molecular receptive range of OR8D1 agonists according to potency and efficacy, and highlights the most active agonists, wherein a fenugreek tonality is common.

FIG. 10 shows the molecular receptive range of OR5AN1 agonists according to potency and efficacy, and highlights the most active agonists, wherein a powdery musk tonality is common.

FIG. 11 shows the molecular receptive range of OR1N2 agonists according to potency and efficacy, and highlights the most active agonists, wherein an animalic musk tonality is common.

FIG. 12 shows the molecular receptive range of OR5A1 agonists according to potency and efficacy, and highlights the most active agonists, wherein a violet tonality is common.

FIG. 13 shows the molecular receptive range of OR7A17 agonists according to potency and efficacy, and highlights the most active agonists, wherein a blonde wood tonality is common.

FIG. 14 shows the molecular receptive range of OR10J5 agonists according to potency and efficacy, and highlights the most active agonists, wherein a muguet tonality is common.

FIG. 15 shows the molecular receptive range of OR1C1 agonists according to potency and efficacy, and highlights the most active agonists, wherein a linalic tonality is common.

FIG. 16 shows the molecular receptive range of OR5B12 agonists according to potency and efficacy, and highlights the most active agonists, wherein a jasmine tonality is common.

FIG. 17 shows the percent activity levels of the ingredients activating OR8D1, OR8B3 and OR5AU1.

FIG. 18 shows the odorant receptor activity induced by accords A and B validation by comparing the model and the in vitro control experiment.

FIG. 19 shows the congruency between perfumery evaluation for the celery tonality and the sensory prediction from the model.

FIG. 20 shows the odorant receptor activity induced by accords A, C and D validation by comparing the model and the in vitro control experiment.

FIG. 21 shows the congruency between perfumery evaluation for the hay tonality and the sensory prediction from the model.

FIG. 22 shows the congruency between perfumery evaluation for the coconut tonality and the sensory prediction from the model.

### DETAILED DESCRIPTION

### Definitions

As used herein, the term “OR” or “olfactory receptor” or “odorant receptor” refers to one or more members of a family of G protein-coupled receptors (GPCRs) that are expressed in olfactory cells. OSNs (olfactory receptor cells) can also be identified on the basis of morphology or by the expression of proteins specifically expressed in olfactory cells. OR family members may have the ability to act as receptors for olfactory signal transduction.

An “agonist of an OR” or “agonist compound” refers to a volatile compound or a ligand that binds to an OR, activates the OR and induces an olfactory receptor transduction cascade.

“Potency” refers to the measure of the activity of a receptor induced by the binding of an agonist (odorant) in terms of amount or concentration required to produce a given activity level. It indicates the sensitivity of the receptor to different agonist concentrations and is often assessed by calculating the EC₅₀ (the agonist concentration necessary to achieve half-maximal receptor activity).

“Efficacy” refers to the measure of the intensity by which the OR responds to a given agonist and is obtained by measuring the activation span between constitutive (baseline in the absence of an agonist) and agonist induced activity.

The “receptive field” or “molecular receptive range” of an odorant receptor refers to the range of volatile compounds activating the receptors. It describes the set of distinct volatile compounds able to bind to the receptor, trigger a transduction cascade within a cell, and hence transfer the chemical stimulus through the olfactory sensory neuron to the brain.

OR or odorant receptor or olfactory receptor polypeptides are considered as such if they pertain to the 7-transmembrane-domain G protein-coupled receptor superfamily encoded by a single approximately 1 kb long exon and exhibit characteristic olfactory receptor-specific amino acid motifs. The seven domains are called “transmembrane” or “TM” domains TM I to TM VII connected by three “internal cellular loop” or “IC” domains IC I to IC III, and three “external cellular loop” or “EC” domains EC I to EC III. The motifs and the variants thereof are defined as, but not restricted to, the MAYDRYVAIC motif (SEQ ID NO: 7) overlapping TM III and IC II, the FSTCSSH motif (SEQ ID NO: 8) overlapping IC III and TM VI, the PMLNPFIY motif (SEQ ID NO: 9) in TM VII as well as three conserved C residues in EC II, and the presence of highly conserved GN residues in TM I [Zhang, X. & Firestein, S. Nat. Neurosci. 5, 124-133 (2002); Malnic, B., et al. Proc. Natl. Acad. Sci. U.S.A 101, 2584-2589 (2004)].

Class I and Class II ORs refer to phylogenetically distinct classes of odorant receptor GPCR. Class I ORs are more related to ORs predominant in aquatic species. Class II ORs are more related to terrestrial species. Mammals carry both types.

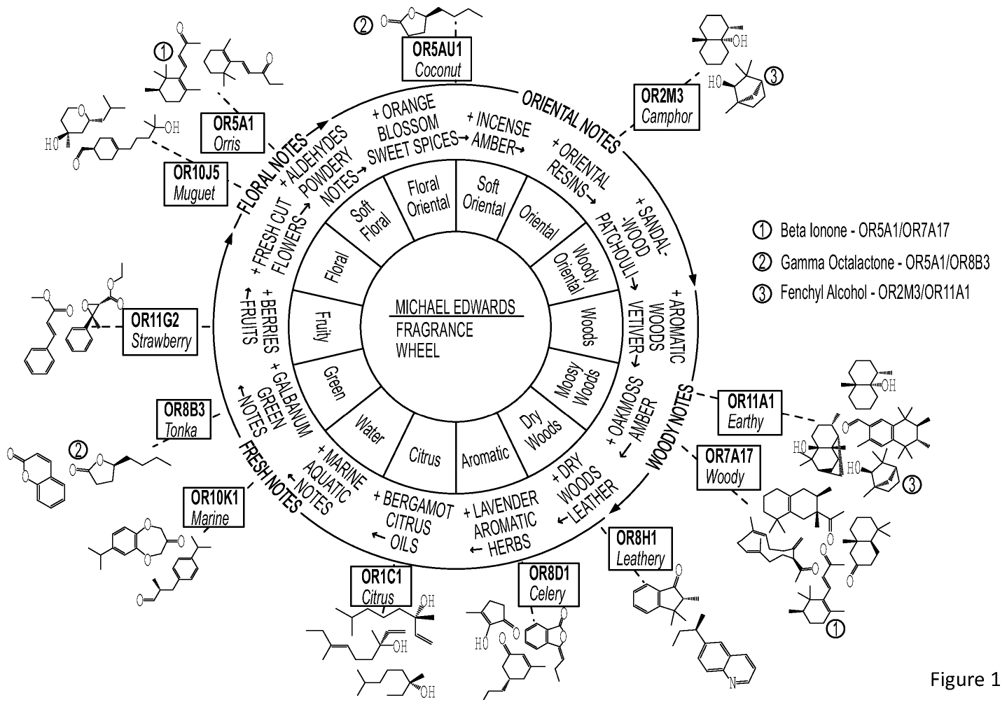

As used herein, “olfactory tonality” or “tonality” means a specific olfactory perception and relates to the activation of an OR by a compound. Non-limiting examples of an olfactory tonality include citrus, coconut, patchouli, etc. Compounds may have greater than one tonality. For example, as shown in FIG. 1, Compound (1) (beta ionone) has violet and blonde woody tonalities arising from the activation of OR5A1 and OR7A17, respectively.

As used herein, a “fragrance” refers to the olfactory perception resulting from the sum of olfactory receptor(s) activation and inhibition (when present) by at least one compound. Accordingly, by way of illustration and by no means intending to limit the scope of the present disclosure, a “fragrance” results from the olfactory perception arising from the sum of a first compound that activates an OR associated with a coconut tonality, a second compound that activates an OR associated with a celery tonality, and a third compound that inhibits an OR associated with a camphor tonality.

As used herein, a “note” or “olfactory note” or “perfumery note” identifies a tonality category. For example, as shown in FIG. 1, floral notes include muguet and violet tonalities.

OR nucleic acids encode a family of GPCRs with seven transmembrane regions that have G protein-coupled receptor activity, e.g., they may bind to G proteins in response to extracellular stimuli and promote production of intracellular second messengers such as IP₃, cAMP, cGMP, and Ca²⁺ via stimulation of enzymes such as phospholipase C and adenylate cyclase III.

The “N terminal domain” region starts at the N-terminus (amino terminus) of a peptide or protein and extends to a region close to the start of the first transmembrane region.

“Transmembrane regions” comprise the seven “transmembrane domains,” which refers to the domain of OR polypeptides that lies within the plasma membrane, and may also include the corresponding cytoplasmic (intracellular) and extracellular loops. The seven transmembrane regions and extracellular and cytoplasmic loops can be identified using standard methods such as hydrophobicity profiles, or as described in Kyte & Doolittle, J. Mol. Biol., 157:105-32 (1982), or in Stryer. The general secondary and tertiary structure of transmembrane domains, in particular the seven transmembrane domains of G protein-coupled receptors such as olfactory receptors, are known in the art. Thus, primary structure sequence can be predicted based on known transmembrane domain sequences. These transmembrane domains are useful for in vitro ligand-binding assays.

The phrase “functional effects” in the context of assays for testing compounds that modulate OR family member mediated olfactory transduction includes the determination of any parameter that is indirectly or directly under the influence of the receptor, e.g., functional, physical and chemical effects. It includes ligand binding, changes in ion flux, membrane potential, current flow, transcription, G protein binding, GPCR phosphorylation or dephosphorylation, signal transduction receptor-ligand interactions, second messenger concentrations (e.g., cAMP, cGMP, IP3, or intracellular Ca.²⁺), in vitro, in vivo, and ex vivo and also includes other physiologic effects such as increases or decreases of neurotransmitter or hormone release.

By “determining the functional effect” or “confirming the activity” in the context of assays is meant assays for a compound that increases or decreases a parameter that is indirectly or directly under the influence of an OR family member, e.g., functional, physical and chemical effects. Such functional effects can be measured by any means known to those skilled in the art, e.g., changes in spectroscopic characteristics (e.g., fluorescence, absorbance, refractive index), hydrodynamic (e.g., shape), chromatographic, or solubility properties, patch clamping, voltage-sensitive dyes, whole cell currents, radioisotope efflux, inducible markers, oocyte OR gene expression; tissue culture cell OR expression; transcriptional activation of OR genes or activity induced genes such as egr-1 or c-fos; ligand-binding assays; voltage, membrane potential and conductance changes; ion flux assays; changes in intracellular second messengers such as cAMP, cGMP, and inositol triphosphate (IP3); changes in intracellular calcium levels; neurotransmitter release, and the like.

The terms “purified,” “substantially purified,” and “isolated” as used herein refer to the state of being free of other, dissimilar compounds with which the compound of the invention is normally associated in its natural state, so that the “purified,” “substantially purified,” and “isolated” subject comprises at least 0.5%, 1%, 5%, 10%, or 20%, or at least 50% or 75% of the mass, by weight, of a given sample. In one particular embodiment, these terms refer to the compound of the invention comprising at least 95, 96, 97, 98, 99 or 100% of the mass, by weight, of a given sample. As used herein, the terms “purified,” “substantially purified,” and “isolated” “isolated,” when referring to a nucleic acid or protein, of nucleic acids or proteins, also refers to a state of purification or concentration different than that which occurs naturally in the mammalian, especially human body. Any degree of purification or concentration greater than that which occurs naturally in the mammalian, especially human body, including (1) the purification from other associated structures or compounds or (2) the association with structures or compounds to which it is not normally associated in the mammalian, especially human, body, are within the meaning of “isolated.” The nucleic acid or protein or classes of nucleic acids or proteins, described herein, may be isolated, or otherwise associated with structures or compounds to which they are not normally associated in nature, according to a variety of methods and processes known to those of skill in the art.

The term “nucleic acid” or “nucleic acid sequence” refers to a deoxy-ribonucleotide or ribonucleotide oligonucleotide in either single- or double-stranded form. The term encompasses nucleic acids, i.e., oligonucleotides, containing known analogs of natural nucleotides. The term also encompasses nucleic-acid-like structures with synthetic backbones. Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating, e.g., sequences in which the third position of one or more selected codons is substituted with mixed-base and/or deoxyinosine residues.

In addition to the gene sequences shown in the sequences disclosed herein, it will be apparent for the person skilled in the art that variants also include DNA sequence polymorphisms that may exist within a given population, which may lead to changes in the amino acid sequence of the polypeptides disclosed herein. Such genetic polymorphisms may exist in cells from different populations or within a population due to natural allelic variation. Allelic variants may also include functional equivalents.

The terms “polypeptide,” “peptide” and “protein” are used interchangeably herein to refer to a polymer of amino acid residues, whether comprising naturally occurring amino acids or polymers and non-naturally occurring amino acids or polymers. The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

By “a non-human organism” or “a host cell” is meant a non-human organism or a cell that contains a nucleic acid as described herein or an expression vector and supports the replication or expression of the expression vector. Host cells may be prokaryotic cells such as E. coli, or eukaryotic cells such as yeast, insect, amphibian, or mammalian cells such as CHO, HeLa, HEK-293, and the like, e.g., cultured cells, explants, and cells in vivo.

By “tag” or “tag combination” is meant a short polypeptide sequence that can be added to the odorant receptor protein. Typically, the DNA encoding a “tag” or a “tag combination” is added to the DNA encoding the receptor, eventually resulting in a fusion protein where the “tag” or a “tag combination” is fused to the N-terminus or C-terminus of the receptor. Lucy, FLAG® and/or Rho tags can enhance the receptor trafficking to the cell membrane, hence the can assist in expression of a functional odorant receptor for in vitro cell based assay [Shepard, B. et al. PLoS One 8, e68758-e68758 (2013), and Zhuang, H. & Matsunami, H. J. Biol. Chem. 282, 15284-15293 (2007)].

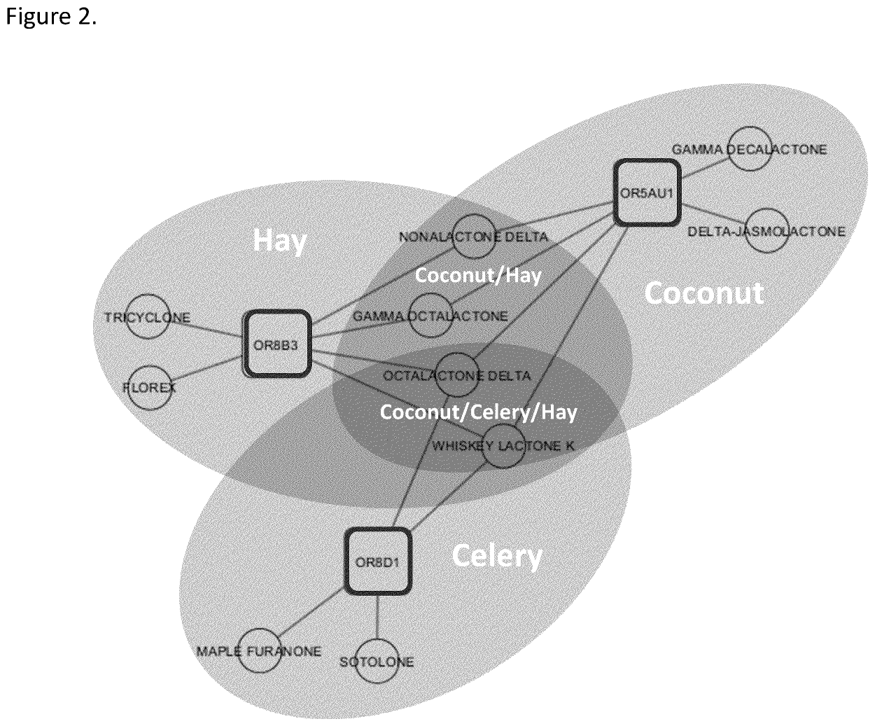

Referring to FIGS. 2 to 16, the present invention includes methods to identify commonalities in olfactory perception between chemically and organoleptically diverse volatile compounds based on their OR activation profile. Without intending to be limited to any particular theory, OR activation profiles represent better predictors of olfactory tonality for a given volatile compound, compared to physicochemical similarities alone. Without intending to be limited to any particular theory, physiochemical similarity may only partially predict olfactory similarity.

The present invention includes methods that can associate at least one olfactive tonality with an OR by screening chemically and organoleptically diverse libraries of volatile compounds against the OR to identify the at least one olfactive tonality common to the volatile compounds that are activators of the screened ORs.

Accordingly, in one aspect, the present invention provides a method for associating at least one olfactory tonality to an olfactory receptor by providing an OR, contacting the OR and a volatile compound having a known at least one tonality, determining whether the compound activates the at least one olfactory receptor, repeating the contacting and determining steps with different compounds having known at least one tonality, categorizing a subset of compounds that activate the OR, identifying an at least one known tonality common to the subset of compounds, and associating the identified at least one known tonality to the OR.

In another aspect, a method is provided, which includes contacting the OR associated with the identified at least one known tonality with at least one volatile compound, determining whether the at least one compound activates the OR, and associating the identified at least one known tonality associated with the OR to the at least one volatile compound if the at least one volatile compound activates the OR.

In some aspects of the present disclosure, methods are used to decode the olfactory code for specific olfactive tonalities, by assessing olfactory receptor activity induced by at least one volatile compound, and associating resulting olfactive tonalities based on the observed olfactive receptor activity.

The plurality of olfactory tonalities of a volatile compound having more than one olfactory tonality can be inferred from the activation of multiple olfactory receptors by the volatile compound. Further, according to the present invention, the plurality of olfactory tonalities of a mixture of volatile compounds can be inferred from the activation of multiple olfactory receptors by the mixture. In some aspects, the mixture comprises more than one volatile compound.

In one aspect, a volatile compound exhibits several olfactive tonalities that can be predicted by assessing olfactory receptor activity induced by the compound, and associating the resulting olfactive tonalities based on its activity on multiple ORs.

In another aspect, the tonality linked to the activity of an OR can be associated by probing the receptor's receptive range, i.e. generate functional activation data to make a comprehensive list of its activators and then identify the most common tonality among them.

In a further aspect, the common olfactive tonality of compounds activating a given receptor is associated by comparing the overall description of the compounds and identifying the descriptor that is common among activators. It should be understood that such an olfactive tonality can be semantically described, e.g., by a perfumer, in several ways. Examples of such semantic similarities capturing similar olfactive tonality include for example: marine, watery and ozone notes; earthy, humus and moss notes; hay, coumarinic and tonka; celery, fenugreek and maple; muguet and lily-of-the-valley.

In one aspect, a method is provided for associating an at least one olfactory tonality to an olfactory receptor comprising:
(a) providing an olfactory receptor;
(b) contacting the olfactory receptor and a compound having at least one known tonality;
(c) determining whether the compound activates the olfactory receptor;
(d) repeating steps (b) and (c) with a compound having at least one known tonality, wherein the compound of step (d) is different than the compound of the preceding iterations of steps (b) and (c);
(e) categorizing as a subset, the compounds from steps (b) through (d) that activate the olfactory receptor;
(f) identifying an at least one tonality common to the subset of compounds; and
(g) assigning the identified at least one tonality to the olfactory receptor.

In one aspect, a method is provided for screening at least one compound having a particular tonality comprising:
(a) providing an olfactory receptor having the identified at least one tonality of a method described according to an aspect presented herein;
(b) contacting the olfactory receptor with an at least one compound;
(c) determining whether the at least one compound activates the olfactory receptor;
(d) associating the at least one compound to the identified at least one tonality if the at least one compound activates the olfactory receptor.

In some aspects, a plurality of olfactory receptors, each having a different identified at least one tonality, is provided in step (a) and a plurality of identified tonalities is associated in step (d) to the compound or combination of compounds that activate the plurality of olfactory receptors according to steps (b) and (c) in the aspect described above.

In one aspect, a method is provided for screening at least one compound for an earthy tonality comprising:
(a) providing a polypeptide comprising an amino acid sequence having at least 90% sequence identity to SEQ ID NO:11;
(b) contacting the polypeptide with the at least one compound;
(c) determining whether the at least one compound activates the polypeptide; and
(d) associating an earthy tonality to the at least one compound if the at least one compound activates the polypeptide;
wherein the polypeptide is an olfactory receptor.

In one aspect, a method is provided for screening at least one compound for a coumarinic tonality comprising:
(a) providing a polypeptide comprising an amino acid sequence having at least 90% sequence identity to SEQ ID NO:13;
(b) contacting the polypeptide with the at least one compound;
(c) determining whether the at least one compound activates the polypeptide; and
(d) associating a coumarinic tonality to the at least one compound if the at least one compound activates the polypeptide;
wherein the polypeptide is an olfactory receptor.

In one aspect, a method is provided for screening at least one compound for a lactonic coconut tonality comprising:
(a) providing a polypeptide comprising an amino acid sequence having at least 90% sequence identity to SEQ ID NO:15;
(b) contacting the polypeptide with the at least one compound;
(c) determining whether the at least one compound activates the polypeptide; and
(d) associating a lactonic coconut tonality to the at least one compound if the at least one compound activates the polypeptide;
wherein the polypeptide is an olfactory receptor.

In one aspect, a method is provided for screening at least one compound for a fenugreek tonality comprising:
(a) providing a polypeptide comprising an amino acid sequence having at least 90% sequence identity to SEQ ID NO:17;
(b) contacting the polypeptide with the at least one compound;
(c) determining whether the at least one compound activates the polypeptide; and
(d) associating a fenugreek tonality to the at least one compound if the at least one compound activates the polypeptide;
wherein the polypeptide is an olfactory receptor.

In one aspect, a method is provided for screening at least one compound for a powdery musk tonality comprising:
(a) providing a polypeptide comprising an amino acid sequence having at least 90% sequence identity to SEQ ID NO:19;
(b) contacting the polypeptide with the at least one compound;
(c) determining whether the at least one compound activates the polypeptide; and
(d) associating a powdery musk tonality to the at least one compound if the at least one compound activates the polypeptide;
wherein the polypeptide is an olfactory receptor.

In one aspect, a method is provided for screening at least one compound for an animalic musk tonality comprising:
(a) providing a polypeptide comprising an amino acid sequence having at least 90% sequence identity to SEQ ID NO:21;
(b) contacting the polypeptide with the at least one compound;
(c) determining whether the at least one compound activates the polypeptide; and
(d) associating an animalic musk tonality to the at least one compound if the at least one compound activates the polypeptide;
wherein the polypeptide is an olfactory receptor.

In one aspect, a method is provided for screening at least one compound for a violet tonality comprising:
(a) providing a polypeptide comprising an amino acid sequence having at least 90% sequence identity to SEQ ID NO:23;
(b) contacting the polypeptide with the at least one compound;
(c) determining whether the at least one compound activates the polypeptide; and
(d) associating a violet tonality to the at least one compound if the at least one compound activates the polypeptide;
wherein the polypeptide is an olfactory receptor.

In one aspect, a method is provided for screening at least one compound for a blonde wood tonality comprising:
(a) providing a polypeptide comprising an amino acid sequence having at least 90% sequence identity to SEQ ID NO:25;
(b) contacting the polypeptide with the at least one compound;
(c) determining whether the at least one compound activates the polypeptide; and
(d) associating a blonde wood tonality to the at least one compound if the at least one compound activates the polypeptide;
wherein the polypeptide is an olfactory receptor.

In one aspect, a method is provided for screening at least one compound for a muguet tonality comprising:
(a) providing a polypeptide comprising an amino acid sequence having at least 90% sequence identity to SEQ ID NO:27;
(b) contacting the polypeptide with the at least one compound;
(c) determining whether the at least one compound activates the polypeptide; and
(d) associating a muguet tonality to the at least one compound if the at least one compound activates the polypeptide;
wherein the polypeptide is an olfactory receptor.

In one aspect, a method is provided for screening at least one compound for a linalic tonality comprising:
(a) providing a polypeptide comprising an amino acid sequence having at least 90% sequence identity to SEQ ID NO:29;
(b) contacting the polypeptide with the at least one compound;
(c) determining whether the compound or combination of compounds activates the polypeptide; and
(d) associating a linalic tonality to the at least one compound if the at least one compound activates the polypeptide;
wherein the polypeptide is an olfactory receptor.

In one aspect, a method is provided for screening at least one compound for a jasmine tonality comprising:
(a) providing a polypeptide comprising an amino acid sequence having at least 90% sequence identity to SEQ ID NO:31;
(b) contacting the polypeptide with the at least one compound;
(c) determining whether the compound or combination of compounds activates the polypeptide; and
(d) associating a jasmine tonality to the at least one compound if the at least one compound activates the polypeptide;
wherein the polypeptide is an olfactory receptor.

In some aspects, the present disclosure provides at least one compound identified by the methods according to certain aspects described herein.

In another aspect, a method is provided for replacing a volatile compound in a composition having a fragrance by selecting a compound in a composition having a fragrance, identifying at least one known tonality of the compound in the composition, providing an OR associated with the at least one known tonality, contacting the OR with a test compound, determining whether the test compound activates the olfactory receptor, and replacing the compound with the identified at least one tonality in the composition with the test compound if the test compound activates the OR.

In another aspect, a method is provided for replacing more than one compound in a composition having a fragrance with a single test compound by identifying the known tonalities of the more than one compound in the composition to be replaced, providing ORs associated with the known tonalities, contacting ORs with the single test compound, and replacing the more than one compound in the composition with the test compound if the test compound activates the same ORs as the more than one compound in the fragrance. As a hypothetical example, a composition having a fragrance includes compound A having a coconut tonality, compound B having a celery tonality, and compound C having a citrus tonality. Test compound X activates ORs associated with coconut and celery tonalities. Test compound X replaces compounds A and B in the composition having a fragrance.

In another aspect, a method is provided for producing a composition having a desired fragrance by choosing a desired fragrance; determining a plurality of tonalities that, in combination, will produce the desired fragrance; and combining one or more compounds that have the plurality of tonalities in a composition.

In a further aspect of the present invention, a method is provided for removing a compound having at least one known tonality in a composition having a desired fragrance by choosing a composition having a desired fragrance, wherein the at least one known tonality is undesirable, or imparts an undesired tonality to the fragrance; identifying an at least one olfactory receptor associated with the at least one known undesired tonality; selecting a compound in the composition having the desired fragrance; contacting the compound and the olfactory receptor; determining whether the compound inhibits the olfactory receptor; and removing the compound from the composition, if the compound inhibits the olfactory receptor.

In a further aspect, a method is provided for adding a compound to a composition having a fragrance by choosing a composition having a fragrance, wherein the added compound inhibits an at least one olfactory receptor associated with the at least one known undesired tonality, and wherein the fragrance is comprised of at least one known tonality; identifying at least one olfactory receptor associated with at least one undesired tonality; contacting the at least one olfactory receptor and a test compound; determining whether the test compound inhibits the olfactory receptor; and adding the test compound to the composition having a fragrance if the test compound inhibits the olfactory receptor.

Methods for adding or replacing a compound to a composition having a fragrance are provided by the present invention according to the following steps: choosing a composition having a fragrance, wherein the fragrance is comprised of at least one tonality; identifying a first olfactory receptor associated with a first of the at least one tonality, wherein the first of the at least one tonality is desired in the fragrance; contacting the first olfactory receptor and a test compound; determining whether the test compound activates the first olfactory receptor; identifying a second olfactory receptor associated with a second of the at least one tonality, wherein the second of the at least one tonality is not desired in the fragrance; contacting the second olfactory receptor and the test compound; determining whether the test compound inhibits the second olfactory receptor; and adding the test compound to the composition or replacing a compound in the composition having a fragrance if the test compound activates the first olfactory receptor and inhibits the second olfactory receptor.

In one aspect, a method is provided for replacing a compound in a composition having a fragrance comprising:
(a) selecting a compound in a composition having a fragrance;
(b) identifying a tonality of the compound in the composition;
(c) providing an olfactory receptor associated with the tonality;
(d) contacting the olfactory receptor and a test compound;
(e) determining whether the test compound activates the olfactory receptor; and
(f) replacing the selected compound in the composition with the test compound if the test compound activates the olfactory receptor.

In one aspect, a method is provided for replacing at least one compound a plurality of compounds in a composition having a fragrance comprising:
(a) selecting a plurality of compounds in a composition having a fragrance, wherein each compound in the plurality has at least one tonality;
(b) identifying the tonalities of the plurality of compounds;
(c) providing a plurality of olfactory receptors, wherein greater than one of the olfactory receptors was assigned the same tonality as at least one of the compounds in the plurality of compounds;
(d) contacting at least one test compound with greater than one of the olfactory receptors;
(e) determining whether the at least one test compound activates greater than one of the olfactory receptors; and
(f) replacing greater than one of the compounds in the plurality of compounds with the test compound if the test compound activates the same olfactory receptors as the greater than one of the compounds in the plurality of compounds.

In one aspect, a method is provided for producing a composition having a desired fragrance comprising:
(a) choosing a desired fragrance;
(b) determining a plurality of tonalities that, in combination, will produce the desired fragrance; and
(c) adding to the composition at least one compound that has the plurality of tonalities.

In some aspects, the at least one compound having the plurality of tonalities is identified according to the methods according to certain aspects described herein.

In one aspect, a method is provided for removing at least one compound in a composition having a desired fragrance comprising:
(a) choosing a composition having a desired fragrance, wherein the fragrance is comprised of at least one tonality;
(b) identifying at least one olfactory receptor associated with the at least one tonality;
(c) selecting at least one compound in the composition having the desired fragrance;
(d) contacting the at least one compound and the at least one olfactory receptor;
(e) determining whether the compound inhibits the at least one olfactory receptor; and
(f) removing the at least one compound from the composition having a desired fragrance if the at least one compound inhibits the at least one olfactory receptor.

In one aspect, a method is provided for adding at least one compound to a composition having a fragrance comprising:
(a) choosing a composition having a fragrance, wherein the fragrance is comprised of at least one tonality;
(b) identifying at least one olfactory receptor associated with the at least one tonality;
(c) contacting the at least one olfactory receptor and at least one test compound;
(d) determining whether the at least one test compound inhibits the at least one olfactory receptor; and
(e) adding the at least one test compound to the composition having a fragrance if the at least one test compound inhibits the at least one olfactory receptor;
wherein the at least one olfactory receptor was assigned a tonality that is not desired in the fragrance.

In one aspect, a method is provided for adding at least one compound to a composition having a fragrance comprising:
(a) choosing a composition having a fragrance, wherein the fragrance is comprised of at least one tonality;
(b) identifying a first olfactory receptor associated with a first of the at least one tonality, wherein the first tonality is desired in the fragrance;
(c) contacting the first olfactory receptor and a test compound;
(d) determining whether the test compound activates the first olfactory receptor;
(e) identifying a second olfactory receptor associated with a second of the at least one tonality, wherein the second tonality is not desired in the fragrance;
(f) contacting the second olfactory receptor and the test compound;
(g) determining whether the test compound inhibits the second olfactory receptor;
and
(h) adding the test compound to the composition having a fragrance if the test compound activates the first olfactory receptor and inhibits the second olfactory receptor.

In some aspects, the added test compound replaces a compound in the fragrance.

The present disclosure further encompasses compounds identified by the methods described herein.

Methods for characterizing relevant receptors and identifying compounds with desired olfactive tonalities that can be used in perfumery or flavor applications to achieve an anticipated perceptual outcome are provided by the present disclosure.

Several alleles may exist for each OR in the human OR repertoire. Each allele comprises distinct single nucleotide polymorphisms (SNPs) that may or may not affect a given OR's receptive range. Thus, OR allelic variations may add or remove compounds and change the absolute and relative potencies and efficacies of compounds in the list of activating compounds and may therefore generate distinct tonality links for each individual OR allele. Similarly, other allelic differences, comprising for example copy number variants (CNVs) or coding sequence truncations, may also lead to changes in OR tonality specificity.

Semantic variability can be addressed by, for example, the collection, curation of, and analysis of multiple sources of perceptual and chemical descriptions of compounds and chemical mixtures, and psychophysical evaluation to determine characteristics of the given compounds and mixtures such as quality, intensity, and associated feelings. The implementation of artificial intelligence algorithms or techniques comprising but not limited to natural language processing, graph convolutional networks, deep neural networks, and other machine learning approaches, as well as statistical analyses of descriptor similarity and/or co-occurrence or potential mutual exclusivity may also be applied to mitigate the confounding effects of semantic differentiation. An automated process can be implemented to associate ORs with a tonality, utilizing probability estimates and correlation scores of descriptor occurrence and measures of receptor activity or binding, including but not limited to potency and efficacy. Clustering of descriptors or compounds or mixtures using various distance measures and clustering methods such as Euclidean distance, earth-movers distance, k-means nearest neighbors, and t-distributed stochastic neighborhood embedding applied to any and all possible metrics of the compounds, mixtures, and descriptors can be used to visualize and validate associations between one or more ORs and one or more tonalities and any and all other properties measurements and features, including, but not limited to, chemical, physical, physicochemical, psychophysical, organoleptic, psychological, emotional, biological and composite properties.

A common olfactive tonality may further be identified by comparing several sources of olfactive descriptions for compounds of interest, such as but not limited to perfumers' descriptions, flavorists' descriptions, trained and untrained panelists' descriptions, publicly available perfumery compound description databases, publicly available flavor compound description databases, F&F industry knowledge and expertise. As used herein, a “perfumer” is an expert in the field of perfumery who can distinguish and describe tonalitiess, whether alone or combined in a fragrance.

In a further embodiment, the tonality linked to the activity of an OR can be identified by specifically testing the receptor with sets of compounds a) with a common tonality but distinct chemical structures and b) with similar chemical structure (e.g. based on Structure-Activity Relationship (SAR) approaches) but distinct tonalities. The resulting comparison between the two sets on the activation of the receptor reveals both chemical requirements for agonism and the common tonality of the agonists of the receptor.

Methods according to the present invention may also be used to characterize the specific tonalities of malodorous compounds. Well-characterized malodor ORs can be screened for compounds that modulate, e.g., enhance, inhibit, allosterically hinder, the malodor OR.

The methods presented herein are not restricted to specific classes of ORs and comprise Class I and Class II ORs, receptors activated by flavor, and perfumery or malodor compounds.

### Methods of Monitoring OR Activity

As long as the function of an OR is not impaired, it may be used in any form in a method or assay described herein. For example, the OR may be used as follows: tissues or cells which intrinsically express an OR such as olfactory sensory neurons isolated from organism and cultured products thereof; olfactory cell membrane bearing the OR; recombinant cells genetically modified so as to express the OR and cultured products thereof; membrane of the recombinant cells; and artificial lipid bilayer membrane carrying the OR.

Indicators for monitoring the activity of olfactory receptors include, for example, a fluorescent calcium indicator dye, a calcium indicator protein (e.g. GCaMP, a genetically encoded calcium indicator), a fluorescent cAMP indicator, a cell mobilization assay, a cellular dynamic mass redistribution assay, a label-free cell based assay, a cAMP response element (CRE) mediated reporter protein, a biochemical cAMP HTRF assay, a beta-arrestin assay, or an electrophysiological recording. In a particular embodiment, a calcium indicator dye is selected that can be used to monitor the activity of olfactory receptors expressed on the membrane of the olfactory neurons (e.g., Fura-2 AM). Compounds may be screened sequentially and the odorant-dependent changes in calcium dye fluorescence are measured using a fluorescent microscope or fluorescent-activated cell sorter (FACS).

As an example, olfactory neurons activated by the target agonist, e.g. a compound with a particular olfactive tonality, are isolated using either a glass microelectrode attached to a micromanipulator or a FACS machine. Mouse olfactory sensory neurons are screened by Ca²⁺ imaging similar to procedures previously described. Malnic, B., et al. Cell 96, 713-723 (1999); Araneda, R. C. et al. J. Physiol. 555, 743-756 (2004); and WO2014/210585. Particularly, a motorized movable microscope stage is used to increase the number of cells that can be screened to at least 1,500 per experiment. Since there are approximately 1,200 different olfactory receptors in the mouse and each olfactory sensory neurons expresses only 1 of 1,200 olfactory receptor genes, this screening capacity will cover virtually the entire mouse odorant receptor repertoire. In other words, the combination of calcium imaging for high-throughput olfactory sensory neuron screening leads to the identification of nearly all of the odorant receptors that respond to a particular profile of odorants. In one aspect, odorant receptors that respond to the target agonist, e.g. with a particular olfactive tonality, can be isolated for receptor identification. For example, at least one neuron is isolated for receptor identification.

Human or non-human mammalian receptors for the target agonist, e.g. with one or more particular olfactive tonalities, may be adapted to a functional assay that can be used to identify compounds that bind, suppress, block, inhibit, and/or modulate the activity of the olfactory receptors. The assay may be a cell-based assay or a binding assay and the method for identifying compounds may be a high-throughput screening assay. More particularly, provided herein are receptor-based assays adaptable for high-throughput screening of receptors with compound libraries for the discovery of positive allosteric modulator, negative allosteric modulators, antagonists or inverse agonist modulator compounds to the particular agonist of interest.

In an embodiment, the target agonist (e.g. having at least one olfactive tonality) receptor gene sequences are identified from the target agonist-sensitive cells as follows. Pooled neurons are heated to 75° C. for 10 minutes to break the cell membrane and render their mRNA available for amplification. This amplification step is important when applying NGS technologies with limited amount of starting material, typically between 1 to 15 cells. Multiple amplification protocols exist. For example, a linear amplification according to the Eberwine method (IVT) ensures the maintenance of the relative transcription levels of expressed genes. Two consecutive overnight (14 h) rounds of in vitro transcription are used to yield sufficient amounts of cRNA; Amplified cRNA is then used to generate an Illumina HiSeq cDNA library. The resulting short sequences of typically 75 to 150 base pairs (commonly referred to as “reads”) are aligned against the reference genome of the mouse (such as UCSC version mm9 or mm10) in order to build the full transcriptome of these cells. Quantitative analysis of the transcriptome data yields a list of transcribed odorant receptor genes and their respective expression levels. Odorant receptor genes that show the most abundant levels of mRNA (most abundant “reads”) or are present in more than one replicate experiment are considered putative target agonist receptors.

The predicted mouse OR genes are then used to mine mouse and human genome databases in order to identify the most closely related receptors (i.e. highest sequence similarity) in mouse (paralogous genes) and in human (orthologous genes). This process may be performed using the BLAST search algorithm (publically available at the NCBI website), a sequence similarity search tool, where every putative gene sequence previously obtained from the initial transcriptome analysis is used as a query sequence. The newly identified genes identified from this data mining process are considered to be potential receptors for a particular olfactive tonality under the assumption that paralogous and orthologous genes are highly likely to possess similar activities. In a particular embodiment, pairwise comparison of sequence homology is carried out to identify closely related receptors in mouse and humans and the receptors are identified as described in WO2014/210585. Other approaches may also be used such as RT-PCR and microarray or mass spectrometry approaches.

In a further embodiment, to complete the deorphanization process, the candidate OR genes are further expressed in vitro for confirmation of activity against the compounds used to isolate the olfactory sensory, or their human orthologs identified as described in a previous embodiment, identified as responding to the target agonist (e.g. having one or more particular olfactive tonalities), and modified at their N-terminus with short polypeptide sequences (e.g., FLAG® (SEQ ID NO: 2), Rho (SEQ ID NO: 4; 20 first amino acids of the bovine rhodopsin receptor), and/or Lucy (SEQ ID NO: 6; cleavable leucine-rich signal peptide sequence) tags), transiently expressed in HEK 293T cells, and stimulated separately with the target agonist (e.g. having a particular olfactive tonality) to confirm their identity as bona fide receptors of the particular target agonist. In a further embodiment, an RTP1 gene can also be expressed in the cell lines whether through activation of the endogenous RTP1 gene, as described in WO 2016201153 A1, or through transformation or viral transduction. Co-expression of the human G alpha subunit Gα_{olf} in this cell-based assay activates the Gs transduction pathway that leads to an internal cAMP increase upon binding to the appropriate ligand. Alternatively, co-expression of the human G alpha subunit Gα15 in the cell based assay activates the Gq transduction pathway that leads to an internal Ca²⁺ increase upon binding to the appropriate ligand.

In a further embodiment, a compound is contacted to an OR, or a chimera or fragment thereof, wherein the OR or a chimera or fragment thereof is expressed in a cell that is recombinantly modified to express the OR, or a chimera or fragment thereof,

In a further embodiment, molecular 3D receptor modeling of ORs is used to assess the binding potential in silico and to identify compounds that may activate, mimic, block, inhibit, modulate, and/or enhance the activity of an OR. In a further embodiment, machine learning algorithms known to those skilled in the art, such as support vector machines, random forests, XGBoost, graph convolutional networks, recurrent neural networks, variational autoencoders, generative adversarial networks and others can be combined with OR activity data, molecular 3D receptor structure data, molecular 3D compound structure data, and physicochemical data, to assess the binding potential in silico and predict compounds that may activate, mimic, block, inhibit, modulate, and/or enhance the activity of an OR.

The activity of the compound can be determined using in vivo, ex vivo, in vitro and synthetic screening systems.

In an embodiment, the contacting may be performed with liposomes or virus-induced budding membranes containing the polypeptides described herein.

In another embodiment, the methods for identifying compounds that bind, suppress, block, inhibit, and/or modulate the activity of an OR may be performed on intact cells or a membrane fraction from cells expressing the polypeptides described here.

An OR described herein may be used for the identification of modulating compounds such as inhibitors or antagonists that would reduce the perception of the particular olfactive tonality associated with the OR.

In a further embodiment, the use of antagonists for an OR encoding a particular olfactory tonality can be used to reveal other, residual, tonalities of a compound, or mixture of multiple compounds. Human ability to consciously and concurrently attend to different olfactory tonalities is known to be limited (e.g. Keller, A (2011)). In the experience of smelling an odor or aroma that possesses multiple tonalities, attention shifts between the tonalities so that only one tonality is attended to at a time. Without wishing to be limited to any particular theory of attentional regulation, the suppression of the tonality encoded by the antagonized OR would necessarily remove it from the pool of tonalities competing for attention, allowing residual tonalities to occupy the attentional space instead. In some cases this relative, rather than absolute, change in the activity of the residual OR leads to the apparent perceptual enhancement of the tonality it encodes.

### Polypeptides Applicable According to the Invention

The present invention includes functional equivalents or analogs or functional mutations of the OR polypeptides specifically described herein.

Functional equivalents refer to polypeptides which, in a test used for OR activity, display at least a 1 to 10%, or at least 20%, or at least 50%, or at least 75%, or at least 90%, or at least 95% higher or lower OR activity.

Functional equivalents, according to the present invention, also cover particular mutants, which, in at least one sequence position of the amino acid sequences stated herein, have an amino acid that is different from that concretely stated, but nevertheless possess one of the aforementioned biological activities. Functional equivalents thus include mutants obtainable by one or more, like 1 to 20, 1 to 15 or 5 to 10 amino acid additions, substitutions, in particular conservative substitutions, deletions and/or inversions, where the stated changes can occur in any sequence position, provided they lead to a mutant with the profile of properties according to the invention. Functional equivalence is in particular also provided if the activity patterns coincide qualitatively between the mutant and the unchanged polypeptide, i.e. if, for example, interaction with the same agonist or antagonist, however at a different rate, (i.e. expressed by an EC₅₀ or IC₅₀ value or any other parameter suitable in the present technical field) is observed. Examples of suitable (conservative) amino acid substitutions are shown in the following table:

| Original residue | Examples of substitution |
|---|---|
| Ala | Ser |
| Arg | Lys |
| Asn | Gln; His |
| Asp | Glu |
| Cys | Ser |
| Gln | Asn |
| Glu | Asp |
| Gly | Pro |
| His | Asn; Gln |
| Ile | Leu; Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Functional equivalents in the above sense also include precursors of the polypeptides described, as well as functional derivatives and salts of the polypeptides. Precursors are natural or synthetic precursors of the polypeptides with or without the desired biological activity.

The expression “salts” means salts of carboxyl groups as well as salts of acid addition of amino groups of the protein molecules according to the invention. Salts of carboxyl groups can be produced in a known way and comprise inorganic salts, for example sodium, calcium, ammonium, iron and zinc salts, and salts with organic bases, for example amines, such as triethanolamine, arginine, lysine, piperidine and the like. The present invention includes salts of acid addition, for example salts with inorganic acids, such as hydrochloric acid or sulfuric acid and salts with organic acids, such as acetic acid and oxalic acid.

Functional derivatives of polypeptides according to the invention can be produced on functional amino acid side groups or at their N-terminal or C-terminal end using known techniques. Such derivatives include, for example, aliphatic esters of carboxylic acid groups, amides of carboxylic acid groups, obtainable by reaction with ammonia or with a primary or secondary amine; N-acyl derivatives of free amino groups, produced by reaction with acyl groups; or O-acyl derivatives of free hydroxyl groups, produced by reaction with acyl groups.

Functional equivalents also comprise polypeptides that can be obtained from other organisms, as well as naturally occurring variants. For example, areas of homologous sequence regions can be established by sequence comparison, and equivalent polypeptides can be determined on the basis of the concrete parameters of the invention.

Functional equivalents also comprise fragments, preferably individual domains or sequence motifs, of the polypeptides according to the invention, which for example display the desired biological function.

Functional equivalents include fusion proteins, which have one of the polypeptide sequences stated herein or functional equivalents derived therefrom and at least one further, functionally different, heterologous sequence in functional N-terminal or C-terminal association (i.e. without substantial mutual functional impairment of the fusion protein parts). Non-limiting examples of these heterologous sequences are e.g. signal peptides, histidine anchors or enzymes.

Functional equivalents in accordance with the present invention include homologs to the specifically disclosed polypeptides. These have at least 60%, preferably at least 75%, in particular at least 80 or 85%, such as, for example, 90, 91, 92, 93, 94, 95, 96, 97, 98 or 99%, homology (or identity) to one of the specifically disclosed amino acid sequences, calculated by the algorithm of Pearson and Lipman, Proc. Natl. Acad, Sci. (USA) 85(8), 1988, 2444-2448. A homology or identity, expressed as a percentage, of a homologous polypeptide according to the invention means in particular an identity, expressed as a percentage, of the amino acid residues based on the total length of one of the amino acid sequences described specifically herein.

The identity data, expressed as a percentage, may also be determined with the aid of BLAST alignments, algorithm blastp (protein-protein BLAST), or by applying the Clustal settings specified herein below.

In the case of a possible protein glycosylation, functional equivalents according to the present invention include the polypeptides described herein in deglycosylated or glycosylated form as well as modified forms that can be obtained by altering the glycosylation pattern.

Such functional equivalents or homologues of the polypeptides according to the invention can be produced by mutagenesis, e.g. by point mutation, lengthening or shortening of the protein or as described in more detail below.

Functional equivalents or homologues of the polypeptides according to the present invention can be identified by screening combinatorial databases of mutants, for example shortening mutants. For example, a variegated database of protein variants can be produced by combinatorial mutagenesis at the nucleic acid level, e.g. by enzymatic ligation of a mixture of synthetic oligonucleotides. There are many methods that can be used for the production of databases of potential homologues from a degenerate oligonucleotide sequence. Chemical synthesis of a degenerate gene sequence can be carried out in an automatic DNA synthesizer, and the synthetic gene can then be ligated in a suitable expression vector. The use of a degenerate gene makes it possible to supply all sequences in a mixture, which code for the desired set of potential protein sequences. Methods of synthesis of degenerate oligonucleotides are known to a person skilled in the art (e.g. Narang, S. A. (1983); Itakura et al. (1984) (a); Itakura et al., (1984) (b); Ike et al. (1983)). Generation of polypeptides with codon-optimized nucleic acid sequences, i.e. adapted to the codon usage frequency of the host cell, are also covered under this method.

Several techniques are known for the screening of gene products of combinatorial databases, which were produced by point mutations or shortening, and for the screening of cDNA libraries for gene products with a selected property. These techniques can be adapted for the rapid screening of the gene banks that were produced by combinatorial mutagenesis of homologues according to the invention. The techniques most frequently used for the screening of large gene banks, which are based on a high-throughput analysis, comprise cloning of the gene bank in expression vectors that can be replicated, transformation of the suitable cells with the resultant vector database and expression of the combinatorial genes in conditions in which detection of the desired activity facilitates isolation of the vector that codes for the gene whose product was detected. Recursive Ensemble Mutagenesis (REM), a technique that increases the frequency of functional mutants in the databases, can be used in combination with the screening tests, in order to identify homologues (Arkin and Yourvan (1992); Delgrave et al. (1993)).

### Nucleic Acid Sequences According to the Invention

The invention includes nucleic acid sequences that code for polypeptides of the present invention.

The present invention also relates to nucleic acids with a certain degree of “identity” to the sequences specifically disclosed herein. “Identity” between two nucleic acids means identity of the nucleotides, in each case over the entire length of the nucleic acid.

For example the identity may be calculated by means of the Vector NTI Suite 7.1 program of the company Informax (USA) employing the Clustal Method (Higgins D G, Sharp P M. ((1989))) with the following settings:

Multiple alignment parameter:

| | |
|---|---|
| Gap opening penalty | 10 |
| Gap extension penalty | 10 |
| Gap separation penalty range | 8 |
| Gap separation penalty | off |
| % identity for alignment delay | 40 |
| Residue specific gaps | off |
| Hydrophilic residue gap | off |
| Transition weighing | 0 |

Pairwise alignment parameter:

| | |
|---|---|
| FAST algorithm | on |
| K-tuple size | 1 |
| Gap penalty | 3 |
| Window size | 5 |
| Number of best diagonals | 5 |

Alternatively the identity may be determined according to Chenna, et al. (2003), the web page: http://www.ebi.ac.uk/Tools/clustalw/index.html# and the following settings:

| | |
|---|---|
| DNA Gap Open Penalty | 15.0 |
| DNA Gap Extension Penalty | 6.66 |
| DNA Matrix | Identity |
| Protein Gap Open Penalty | 10.0 |
| Protein Gap Extension Penalty | 0.2 |
| Protein matrix | Gonnet |
| Protein/DNA ENDGAP | −1 |
| Protein/DNA GAPDIST | 4 |

The invention also relates to nucleic acid sequences (single-stranded and double-stranded DNA and RNA sequences, e.g. cDNA and mRNA), coding for one of the above polypeptides and their functional equivalents, which can be obtained, for example, using artificial nucleotide analogs.

The present invention relates both to isolated nucleic acid molecules, which code for polypeptides according to the invention, or biologically active segments thereof, and to nucleic acid fragments, which can be used for example as hybridization probes or primers for identifying or amplifying coding nucleic acids according to the invention.

The nucleic acid molecules according to the invention can in addition contain non-translated sequences from the 3′ and/or 5′ end of the coding genetic region. The invention further relates to the nucleic acid molecules that are complementary to the concretely described nucleotide sequences or a segment thereof.

The nucleotide sequences according to the invention make possible the production of probes and primers that can be used for the identification and/or cloning of homologous sequences in other cellular types and organisms. Probes or primers generally comprise a nucleotide sequence region which hybridizes under “stringent” conditions (see below) on at least about 12, preferably at least about 25, for example about 40, 50 or 75 successive nucleotides of a sense strand of a nucleic acid sequence according to the invention or of a corresponding antisense strand.

An “isolated” nucleic acid molecule is separated from other nucleic acid molecules that are present in the natural source of the nucleic acid and can moreover be substantially free from other cellular material or culture medium, if it is being produced by recombinant techniques, or can be free from chemical precursors or other chemicals, if it is being synthesized chemically.

“Hybridize” means the ability of a polynucleotide or oligonucleotide to bind to an almost complementary sequence in standard conditions, whereas nonspecific binding does not occur between non-complementary partners in these conditions. For this, the sequences can be 90-100% complementary. The property of complementary sequences of being able to bind specifically to one another is utilized for example in Northern Blotting or Southern Blotting or in primer binding in PCR or RT-PCR.

Short oligonucleotides of the conserved regions are used advantageously for hybridization. However, it is also possible to use longer fragments of the nucleic acids according to the invention or the complete sequences for the hybridization. These standard conditions vary depending on the nucleic acid used (oligonucleotide, longer fragment or complete sequence) or depending on which type of nucleic acid—DNA or RNA—is used for hybridization. For example, the melting temperatures for DNA:DNA hybrids are approx. 10° C. lower than those of DNA:RNA hybrids of the same length.

For example, depending on the particular nucleic acid, standard conditions mean temperatures between 42 and 58° C. in an aqueous buffer solution with a concentration between 0.1 to 5×SSC (1×SSC=0.15 M NaCl, 15 mM sodium citrate, pH 7.2) or additionally in the presence of 50% formamide, for example 42° C. in 5×SSC, 50% formamide. Advantageously, the hybridization conditions for DNA:DNA hybrids are 0.1×SSC and temperatures between about 20° C. to 45° C., preferably between about 30° C. to 45° C. For DNA:RNA hybrids the hybridization conditions are advantageously 0.1×SSC and temperatures between about 30° C. to 55° C., preferably between about 45° C. to 55° C. These stated temperatures for hybridization are examples of calculated melting temperature values for a nucleic acid with a length of approx. 100 nucleotides and a G+C content of 50% in the absence of formamide. The experimental conditions for DNA hybridization are described in relevant genetics textbooks, for example Sambrook et al., 1989, and can be calculated using formulae that are known by a person skilled in the art, for example depending on the length of the nucleic acids, the type of hybrids or the G+C content. A person skilled in the art can obtain further information on hybridization from the following textbooks: Ausubel et al. (eds), (1985), Brown (ed) (1991).

Hybridization can be carried out under stringent conditions. Such hybridization conditions are for example described in Sambrook (1989), or in Current Protocols in Molecular Biology, John Wiley & Sons, N.Y. (1989), 6.3.1-6.3.6.

As used herein, the term hybridization or hybridizes under certain conditions is intended to describe conditions for hybridization and washes under which nucleotide sequences that are significantly identical or homologous to each other remain bound to each other. The conditions may be such that sequences, which are at least about 70%, such as at least about 80%, and such as at least about 85%, 90%, or 95% identical, remain bound to each other.

Appropriate hybridization conditions can be selected by those skilled in the art with minimal experimentation as exemplified in Ausubel et al. (1995, Current Protocols in Molecular Biology, John Wiley & Sons, sections 2, 4, and 6). Additionally, stringency conditions are described in Sambrook et al. (1989, Molecular Cloning: A Laboratory Manual, 2nd ed., Cold Spring Harbor Press, chapters 7, 9, and 11).

Conditions of low stringency are as follows. Filters containing DNA are pretreated for 6 h at 40° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA. Hybridizations are carried out in the same solution with the following modifications: 0.02% PVP, 0.02% Ficoll, 0.2% BSA, 100 μg/ml salmon sperm DNA, 10% (wt/vol) dextran sulfate, and 5-20×106 32P-labeled probe is used. Filters are incubated in hybridization mixture for 18-20 h at 40° C., and then washed for 1.5 h at 55° C. In a solution containing 2×SSC, 25 mM Tris-HCl (pH 7.4), 5 mM EDTA, and 0.1% SDS. The wash solution is replaced with fresh solution and incubated an additional 1.5 h at 60° C. Filters are blotted dry and exposed for autoradiography.

Conditions of moderate stringency are as follows. Filters containing DNA are pretreated for 7 h at 50° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA. Hybridizations are carried out in the same solution with the following modifications: 0.02% PVP, 0.02% Ficoll, 0.2% BSA, 100 μg/ml salmon sperm DNA, 10% (wt/vol) dextran sulfate, and 5-20×106 32P-labeled probe is used. Filters are incubated in hybridization mixture for 30 h at 50° C., and then washed for 1.5 h at 55° C. In a solution containing 2×SSC, 25 mM Tris-HCl (pH 7.4), 5 mM EDTA, and 0.1% SDS. The wash solution is replaced with fresh solution and incubated an additional 1.5 h at 60° C. Filters are blotted dry and exposed for autoradiography.

Conditions of high stringency are as follows. Prehybridization of filters containing DNA is carried out for 8 h to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65° C. in the prehybridization mixture containing 100 μg/ml denatured salmon sperm DNA and 5-20×10⁶ cpm of ³²P-labeled probe. Washing of filters is done at 37° C. for 1 h in a solution containing 2×SSC, 0.01% PVP, 0.01% Ficoll, and 0.01% BSA. This is followed by a wash in 0.1×SSC at 50° C. for 45 minutes.

Other conditions of low, moderate, and high stringency well known in the art (e.g., as employed for cross-species hybridizations) may be used if the above conditions are inappropriate (e.g., as employed for cross-species hybridizations).

Nucleic acid sequences according to the invention can be derived from the sequences specifically disclosed herein and can differ from it by addition, substitution, insertion or deletion of individual or several nucleotides, and furthermore code for polypeptides with the desired profile of properties.

The invention also encompasses nucleic acid sequences that comprise so-called silent mutations or have been altered, in comparison with a concretely stated sequence, according to the codon usage of a special original or host organism, as well as naturally occurring variants, e.g. splicing variants or allelic variants, thereof.

Derivatives of nucleic acid sequences according to the invention mean for example allelic variants, having at least 60% homology at the level of the derived amino acid, preferably at least 80% homology, quite especially preferably at least 90% homology over the entire sequence range (regarding homology at the amino acid level, reference should be made to the details given above for the polypeptides). Advantageously, the homologies can be higher over partial regions of the sequences.

Furthermore, derivatives are also to be understood to be homologues of the nucleic acid sequences according to the invention, for example animal, plant, fungal or bacterial homologues, shortened sequences, single-stranded DNA or RNA of the coding and noncoding DNA sequence. For example, homologues have, at the DNA level, a homology of at least 40%, preferably of at least 60%, especially preferably of at least 70%, quite especially preferably of at least 80% over the entire DNA region given in a sequence specifically disclosed herein.

Moreover, derivatives are to be understood to be, for example, fusions with promoters. The promoters that are added to the stated nucleotide sequences can be modified by at least one nucleotide exchange, at least one insertion, inversion and/or deletion, though without impairing the functionality or efficacy of the promoters. Moreover, the efficacy of the promoters can be increased by altering their sequence or can be exchanged completely with more effective promoters even of organisms of a different genus.

Moreover, a person skilled in the art is familiar with methods for generating functional mutants, that is to say nucleotide sequences which code for a polypeptide with at least 80%, 81%, 82%, 83%, 84%, 85%, 86%, 87%, 88%, 89%, 90%, 91%, 92%, 93%, 94%, 95%, 96%, 97%, 98% or 99% sequence identity to anyone of SEQ ID NO: 2, 4, 6, or 8; and/or encoded by a nucleic acid molecule comprising a nucleotide sequence having at least 70% sequence identity to SEQ ID NO: 1, 3, 5, or 7.

Depending on the technique used, a person skilled in the art can introduce entirely random or else more directed mutations into genes or else noncoding nucleic acid regions (which are for example important for regulating expression) and subsequently generate genetic libraries. The methods of molecular biology required for this purpose are known to the skilled worker and for example described in Sambrook and Russell, Molecular Cloning. 3rd Edition, Cold Spring Harbor Laboratory Press 2001.

Methods for modifying genes and thus for modifying the polypeptide encoded by them have been known to the skilled worker for a long time, such as, for example
site-specific mutagenesis, where individual or several nucleotides of a gene are replaced in a directed fashion (Trower M K (Ed.) 1996; In vitro mutagenesis protocols. Humana Press, New Jersey),
saturation mutagenesis, in which a codon for any amino acid can be exchanged or added at any point of a gene (Kegler-Ebo D M, Docktor C M, DiMaio D (1994) Nucleic Acids Res 22:1593; Barettino D, Feigenbutz M, Valcárel R, Stunnenberg H G (1994) Nucleic Acids Res 22:541; Barik S (1995) Mol Biotechnol 3:1),
error-prone polymerase chain reaction, where nucleotide sequences are mutated by error-prone DNA polymerases (Eckert K A, Kunkel T A (1990) Nucleic Acids Res 18:3739);
the SeSaM method (sequence saturation method), in which preferred exchanges are prevented by the polymerase. Schenk et al., Biospektrum, Vol. 3, 2006, 277-279
   the passaging of genes in mutator strains, in which, for example owing to defective DNA repair mechanisms, there is an increased mutation rate of nucleotide sequences (Greener A, Callahan M, Jerpseth B (1996) An efficient random mutagenesis technique using an E. coli mutator strain. In: Trower M K (Ed.) In vitro mutagenesis protocols. Humana Press, New Jersey), or
   DNA shuffling, in which a pool of closely related genes is formed and digested and the fragments are used as templates for a polymerase chain reaction in which, by repeated strand separation and reassociation, full-length mosaic genes are ultimately generated (Stemmer W P C (1994) Nature 370:389; Stemmer W P C (1994) Proc Natl Acad Sci USA 91:10747).

Using so-called directed evolution (described, inter alia, in Reetz M T and Jaeger K-E (1999), Topics Curr Chem 200:31; Zhao H, Moore J C, Volkov A A, Arnold F H (1999), Methods for optimizing industrial polypeptides by directed evolution, In: Demain A L, Davies J E (Ed.) Manual of industrial microbiology and biotechnology. American Society for Microbiology), a skilled worker can produce functional mutants in a directed manner and on a large scale. To this end, in a first step, gene libraries of the respective polypeptides are first produced, for example using the methods given above. The gene libraries are expressed in a suitable way, for example by bacteria or by phage display systems.

The relevant genes of host organisms which express functional mutants with properties that largely correspond to the desired properties can be submitted to another mutation cycle. The steps of the mutation and selection or screening can be repeated iteratively until the present functional mutants have the desired properties to a sufficient extent. Using this iterative procedure, a limited number of mutations, for example 1, 2, 3, 4 or 5 mutations, can be performed in stages and assessed and selected for their influence on the activity in question. The selected mutant can then be submitted to a further mutation step in the same way. In this way, the number of individual mutants to be investigated can be reduced significantly.

The results according to the invention also provide important information relating to structure and sequence of the relevant polypeptides, which is required for generating, in a targeted fashion, further polypeptides with desired modified properties. In particular, it is possible to define so-called “hot spots”, i.e. sequence segments that are potentially suitable for modifying a property by introducing targeted mutations.

Information can also be deduced regarding amino acid sequence positions, in the region of which mutations can be effected that should probably have little effect on the activity, and can be designated as potential “silent mutations”.

For expression in a suitable host organism, the recombinant nucleic acid construct or gene construct is advantageously inserted into a host-specific vector which makes possible optimal expression of the genes in the host. Vectors are well known to the skilled worker and can be found for example in “cloning vectors” (Pouwels P. H. et al., Ed., Elsevier, Amsterdam-New York-Oxford, 1985).

Nucleic acid and amino acid sequences identified and/or used herein are listed below:

FLAG ® tag
DNA
SEQ ID NO: 1
gattacaaggacgacgacgataag
Protein
SEQ ID NO: 2
DYKDDDDK
Rho tag
DNA
SEQ ID NO: 3
atgaacgggaccgagggcccaaacttctacgtgcctttctccaacaagacgggcgtggtg
Protein
SEQ ID NO: 4
MNGTEGPNFYVPFSNKTGVV
Lucy tag
DNA
SEQ ID NO: 5
atgagaccccagatcctgctgctcctggccctgctgaccctaggcctggct
protein
SEQ ID NO: 6
MRPQILLLLALLTLGLA
Motif
SEQ ID NO: 7
MAYDRYVAIC
Motif
SEQ ID NO: 8
FSTCSSH
Motif
SEQ ID NO: 9
PMLNPFIY
OR11A1
DNA
SEQ ID NO: 10
atggaaattgtctccacaggaaacgaaactattactgaatttgtcctccttggcttctatgacatccctgaactgca
tttcttgttttttattgtattcactgctgtctatgtcttcatcatcatagggaatatgctgattattgtagcagtgg
ttagctcccagaggctccacaaacccatgtatattttcttggcgaatctgtccttcctggatattctctacacctcc
gcagtgatgccaaaaatgctggagggcttcctgcaagaagcaactatctctgtggctggttgcttgctccagttctt
tatcttcggctctctagccacagctgaatgcttactgctggctgtcatggcatatgaccgctacctggcaatttgct
acccactccactacccactcctgatggggcccagacggtacatggggctggtggtcacaacctggctctctggattt
gtggtagatggactggttgtggccctggtggcccagctgaggttctgtggccccaaccacattgaccagttttactg
tgactttatgcttttcgtgggcctggcttgctcggatcccagagtggctcaggtgacaactctcattctgtctgtgt
tctgcctcactattccttttggactgattctgacatcttatgccagaattgtggtggcagtgctgagagttcctgct
ggggcaagcaggagaagggctttctccacatgctcctcccacctagctgtagtgaccacattctatggaacgctcat
gatcttttatgttgcaccctctgctgtccattcccagctcctctccaaggtcttctccctgctctacactgtggtca
cccctctcttcaatcctgtgatctataccatgaggaacaaggaggtgcatcaggcacttcggaagattctctgtatc
aaacaaactgaaacacttgattga
Protein
SEQ ID NO: 11
MEIVSTGNETITEFVLLGFYDIPELHFLFFIVFTAVYVFIIIGNMLIIVAVVSSQRLHKPMYIFLANLSFLDILYTS
AVMPKMLEGFLQEATISVAGCLLQFFIFGSLATAECLLLAVMAYDRYLAICYPLHYPLLMGPRRYMGLVVTTWLSGF
WDGLWALVAQLRFCGPNHIDQFYCDFMLFVGLACSDPRVAQVTTLILSVFCLTIPFGLILTSYARIWAVLRVPA
GASRRRAFSTCSSHLAWTTFYGTLMIFYVAPSAVHSQLLSKVFSLLYTWTPLFNPVIYTMRNKEVHQALRKILCI
KQTETLD
OR8B3
DNA
SEQ ID NO: 12
atgctggctagaaacaactccttagtgactgaatttattcttgctggattaacagatcatccagagttccagcaacc
cctctttttcctgtttctagtggtctacattgtcaccatggtaggcaaccttggcttgatcattcttttcggtctaa
attctcacctccacacaccaatgtactatttcctcttcaatctctccttcattgatctctgttactcctctgttttc
actcccaaaatgctaatgaactttgtatcaaaaaagaatattatctcctatgttgggtgcatgactcagctgttttt
ctttctcttttttgtcatctctgaatgttacatgttgacctcaatggcatatgatcgctatgtggccatctgtaatc
cattgctgtataaggtcaccatgtcccatcaggtctgttctatgctcacttttgctgcttacataatgggattggct
ggagccacggcccacaccgggtgcatgcttagactcaccttctgcagtgctaatatcatcaaccattacttgtgtga
catactccccctcctccagctttcctgcaccagcacctatgtcaacgaggtggttgttctcattgttgtgggtatta
atatcatggtacccagttgtaccatcctcatttcttatgttttcattgtcactagcattcttcatatcaaatccact
caaggaagatcaaaagccttcagtacttgtagctctcatgtcattgctctgtctctgttttttgggtcagcggcatt
catgtatattaaatattcttctggatctatggagcagggaaaagtttcttctgttttctacactaatgtggtgccca
tgctcaatcctctcatctacagtttgaggaacaaggatgtcaaagttgcactgaggaaagctctgattaaaattcag
agaagaaatatattctaa
Protein
SEQ ID NO: 13
MLARNNSLVTEFILAGLTDHPEFQQPLFFLFLVVYIVTMVGNLGLIILFGLNSHLHTPMYYFLFNLSFIDLCYSSVF
TPKMLMNFVSKKNIISYVGCMTQLFFFLFFVISECYMLTSMAYDRYVAICNPLLYKVTMSHQVCSMLTFAAYIMGLA
GATAHTGCMLRLTFCSANIINHYLCDILPLLQLSCTSTYVNEVWLIWGINIMVPSCTILISYVFIVTSILHIKST
QGRSKAFSTCSSHVIALSLFFGSAAFMYIKYSSGSMEQGKVSSVFYTNVVPMLNPLIYSLRNKDVKVALRKALIKIQ
RRNIF
OR5AU1
DNA
SEQ ID NO: 14
atgaaaggggcaaacctgagccaagggatggagtttgagctcttgggcctcaccactgacccccagctccagaggct
gctcttcgtggtgttcctgggcatgtacacagccactctgctggggaacctggtcatgttcctcctgatccatgtga
gtgccaccctgcacacacccatgtactccctcctgaagagcctctccttcttggatttctgctactcctccacggtt
gtgccccagaccctggtgaacttcttggccaagaggaaagtgatctcttattttggctgcatgactcagatgttctt
ctatgcgggttttgccaccagtgagtgctatctcatcgctgccatggcctatgaccgctatgccgctatttgtaacc
ccctgctctactcaaccatcatgtctcctgaggtctgtgcctcgctgattgtgggctcctacagtgcaggattcctc
aattctcttatccacactggctgtatctttagtctgaaattctgcggtgctcatgtcgtcactcacttcttctgtga
tgggccacccatcctgtccttgtcttgtgtagacacctcactgtgtgagatcctgctcttcatttttgctggtttca
accttttgagctgcaccctcaccatcttgatctcctacttcttaattctcaacaccatcctgaaaatgagctcggcc
cagggcaggtttaaggcattttccacctgtgcatcccacctcactgccatctgcctcttctttggcacaacactttt
tatgtacctgcgccccaggtccagctactccttgacccaggaccgcacagttgctgtcatctacacagtggtgatcc
cagtgctgaaccccctcatgtactctttgagaaacaaggatgtgaagaaagctttaataaaggtttggggtaggaaa
acaatggaatga
Protein
SEQ ID NO: 15
MKGANLSQGMEFELLGLTTDPQLQRLLFVVFLGMYTATLLGNLVMFLLIHVSATLHTPMYSLLKSLSFLDFCYSSTV
VPQTLVNFLAKRKVISYFGCMTQMFFYAGFATSECYLIAAMAYDRYAAICNPLLYSTIMSPEVCASLIVGSYSAGFL
NSLIHTGCIFSLKFCGAHVVTHFFCDGPPILSLSCVDTSLCEILLFIFAGFNLLSCTLTILISYFLILNTILKMSSA
QGRFKAFSTCASHLTAICLFFGTTLFMYLRPRSSYSLTQDRTVAVIYTVVIPVLNPLMYSLRNKDVKKALIKVWGRK
TME
OR8D1
DNA
SEQ ID NO: 16
atgaccatggaaaattattctatggcagctcagtttgtcttagatggtttaacacagcaagcagagctccagctgcc
cctcttcctcctgttcctgggaatctatgtggtcacagtagtgggcaacctgggcatgattctcctgattgcagtca
gccctctacttcacacccccatgtactatttcctcagcagcttgtccttcgtcgatttctgctattcctctgtcatt
actcccaaaatgctggtgaacttcctaggaaagaagaatacaatcctttactctgagtgcatggtccagctcttttt
ctttgtggtctttgtggtggctgagggttacctcctgactgccatggcatatgatcgctatgttgccatctgtagcc
cactgctttataatgcgatcatgtcctcatgggtctgctcactgctagtgctggctgccttcttcttgggctttctc
tctgccttgactcatacaagtgccatgatgaaactgtccttttgcaaatcccacattatcaaccattacttctgtga
tgttcttcccctcctcaatctctcctgctccaacacacacctcaatgagcttctactttttatcattgcggggttta
acaccttggtgcccaccctagctgttgctgtctcctatgccttcatcctctacagcatccttcacatccgctcctca
gagggccggtccaaagcttttggaacatgcagctctcatctcatggctgtggtgatcttctttgggtccattacctt
catgtatttcaagcccccttcaagtaactccctggaccaggagaaggtgtcctctgtgttctacaccacggtgatcc
ccatgctgaaccctttaatatacagtctgaggaataaggatgtgaagaaagcattaaggaaggtcttagtaggaaaa
tga
Protein
SEQ ID NO: 17
MTMENYSMAAQFVLDGLTQQAELQLPLFLLFLGIYWTWGNLGMILLIAVSPLLHTPMYYFLSSLSFVDFCYSSVI
TPKMLVNFLGKKNTILYSECMVQLFFFVVFVVAEGYLLTAMAYDRYVAICSPLLYNAIMSSWVCSLLVLAAFFLGFL
SALTHTSAMMKLSFCKSHIINHYFCDVLPLLNLSCSNTHLNELLLFIIAGFNTLVPTLAVAVSYAFILYSILHIRSS
EGRSKAFGTCSSHLMAVVIFFGSITFMYFKPPSSNSLDQEKVSSVFYTTVIPMLNPLIYSLRNKDVKKALRKVLVGK
OR5AN1
DNA
SEQ ID NO: 18
atgactgggggaggaaatattacagaaatcacctatttcatcctgctgggattctcagattttcccaggatcataaa
agtgctcttcactatattcctggtgatctacattacatctctggcctggaacctctccctcattgttttaataagga
tggattcccacctccatacacccatgtatttcttcctcagtaacctgtccttcatagatgtctgctatatcagctcc
acagtccccaagatgctctccaacctcttacaggaacagcaaactatcacttttgttggttgtattattcagtactt
tatcttttcaacgatgggactgagtgagtcttgtctcatgacagccatggcttatgatcgttatgctgccatttgta
accccctgctctattcatccatcatgtcacccaccctctgtgtttggatggtactgggagcctacatgactggcctc
actgcttctttattccaaattggtgctttgcttcaactccacttctgtgggtctaatgtcatcagacatttcttctg
tgacatgccccaactgttaatcttgtcctgtactgacactttctttgtacaggtcatgactgctatattaaccatgt
tctttgggatagcaagtgccctagttatcatgatatcctatggctatattggcatctccatcatgaagatcacttca
gctaaaggcaggtccaaggcattcaacacctgtgcttctcatctaacagctgtttccctcttctatacatcaggaat
ctttgtctatttgagttccagctctggaggttcttcaagctttgacagatttgcatctgttttctacactgtggtca
ttcccatgttaaatcccttgatttacagtttgaggaacaaagaaattaaagatgccttaaagaggttgcaaaagaga
aagtgctgctga
Protein
SEQ ID NO: 19
MTGGGNITEITYFILLGFSDFPRIIKVLFTIFLVIYITSLAWNLSLIVLIRMDSHLHTPMYFFLSNLSFIDVCYISS
TVPKMLSNLLQEQQTITFVGCIIQYFIFSTMGLSESCLMTAMAYDRYAAICNPLLYSSIMSPTLCVWMVLGAYMTGL
TASLFQIGALLQLHFCGSNVIRHFFCDMPQLLILSCTDTFFVQVMTAILTMFFGIASALVIMISYGYIGISIMKITS
AKGRSKAFNTCASHLTAVSLFYTSGIFVYLSSSSGGSSSFDRFASVFYTVVIPMLNPLIYSLRNKEIKDALKRLQKR
KCC
OR1N2
DNA
SEQ ID NO: 20
atgggaaaaccaggcagagtgaaccaaaccactgtttcagacttcctccttttaggactctctgagcggccagagga
gcagcctcttctgtttggcatcttccttggcatgtacctggtcaccatggtggggaacctgctcattatcctggcca
tcagctctgacccacacctccatactcccatgtacttctttctggccaacctgtcattaactgatgcctgtttcact
tctgcctccatccccaaaatgctggccaacattcatacccagagtcagatcatctcctattctgggtgtcttgcaca
gctatatttcctccttatgtttggtggccttgacaactgcctgctggctgtgatggcatatgaccgctatgtggcca
tctgccaaccactccattacagcacatctatgagtccccagctctgtgcactaatgctgggtgtgtgctgggtgcta
accaactgtcctgccctgatgcacacactgttgctgacccgcgtggctttctgtgcccagaaagccatccctcattt
ctactgtgatcccagtgctctcctgaagcttgcctgctcagatacccatgtaaacgagctgatgatcatcaccatgg
gcttgctgttcctcactgttcccctcctgctgatcgtcttctcctatgtccgcattttctgggctgtgtttggcatc
tcatctcctggagggagatggaaggccttctctacctgtggttctcatctcacggtggttctgctcttctatgggtc
tcttatgggtgtgtatttacttcctccatcaacttactctacagagagggaaagtagggctgctgttctctatatgg
tgattattcccatgctaaacccattcatttatagcttgaggaacagagacatgaaggaggctttgggtaaacttttt
gtcagtggaaaaacattctttttatga
Protein
SEQ ID NO: 21
MGKPGRVNQTTVSDFLLLGLSERPEEQPLLFGIFLGMYLVTMVGNLLIILAISSDPHLHTPMYFFLANLSLTDACFT
SASIPKMLANIHTQSQIISYSGCLAQLYFLLMFGGLDNCLLAVMAYDRYVAICQPLHYSTSMSPQLCALMLGVCWVL
TNCPALMHTLLLTRVAFCAQKAIPHFYCDPSALLKLACSDTHVNELMIITMGLLFLTVPLLLIVFSYVRIFWAVFGI
SSPGGRWKAFSTCGSHLTVVLLFYGSLMGVYLLPPSTYSTERESRAAVLYMVIIPMLNPFIYSLRNRDMKEALGKLF
VSGKTFFL
OR5A1
DNA
SEQ ID NO: 22
atgtccataaccaaagcctggaacagctcatcagtgaccatgttcatcctcctgggattcacagaccatccagaact
ccaggccctcctctttgtgaccttcctgggcatctatcttaccaccctggcctggaacctggccctcatttttctga
tcagaggtgacacccatctgcacacacccatgtacttcttcctaagcaacttatctttcattgacatctgctactct
tctgctgtggctcccaatatgctcactgacttcttctgggagcagaagaccatatcatttgtgggctgtgctgctca
gttttttttctttgtcggcatgggtctgtctgagtgcctcctcctgactgctatggcatacgaccgatatgcagcca
tctccagcccccttctctaccccactatcatgacccagggcctctgtacacgcatggtggttggggcatatgttggt
ggcttcctgagctccctgatccaggccagctccatatttaggcttcacttttgcggacccaacatcatcaaccactt
cttctgcgacctcccaccagtcctggctctgtcttgctctgacaccttcctcagtcaagtggtgaatttcctcgtgg
tggtcactgtcggaggaacatcgttcctccaactccttatctcctatggttacatagtgtctgcggtcctgaagatc
ccttcagcagagggccgatggaaagcctgcaacacgtgtgcctcgcatctgatggtggtgactctgctgtttgggac
agcccttttcgtgtacttgcgacccagctccagctacttgctaggcagggacaaggtggtgtctgttttctattcat
tggtgatccccatgctgaaccctctcatttacagtttgaggaacaaagagatcaaggatgccctgtggaaggtgttg
gaaaggaagaaagtgttttcttag
Protein
SEQ ID NO: 23
MSITKAWNSSSVTMFILLGFTDHPELQALLFVTFLGIYLTTLAWNLALIFLIRGDTHLHTPMYFFLSNLSFIDICYS
SAVAPNMLTDFFWEQKTISFVGCAAQFFFFVGMGLSECLLLTAMAYDRYAAISSPLLYPTIMTQGLCTRMVVGAYVG
GFLSSLIQASSIFRLHFCGPNIINHFFCDLPPVLALSCSDTFLSQVVNFLVVVTVGGTSFLQLLISYGYIVSAVLKI
PSAEGRWKACNTCASHLMVVTLLFGTALFVYLRPSSSYLLGRDKVVSVFYSLVIPMLNPLIYSLRNKEIKDALWKVL
ERKKVFS
OR7A17
DNA
SEQ ID NO: 24
atggaaccagagaatgacacagggatttcagaatttgttcttctgggactttctgaggaaccagaattgcagccctt
cctctttgggctgtttctgtccatgtacctggtcactgtgctcgggaatctgctcatcatcctggccacaatctcag
actcccacctccacacccccatgtacttcttcctctccaacctgtcctttgcagacatctgtttcatctccactaca
atcccaaagatgctcattaacatccagacacagagcagagtcatcacctatgcaggctgcatcacccagatgtgctt
ttttgtactttttggagggttagacagcttactcctggctgtgatggcctatgatcggtttgtggccatctgtcatc
ctctgcactacacagtcatcatgaaccctcggctctgtggactcctggttctggcatcctggatgattgctgccctg
aattccttgtcacaaagcttaatggtattgtggctgtccttctgcacagacttggaaatcccccactttttctgtga
acttaatcaggtcatccaccttgcctgttctgacacctttcttaatgacatggggatgtattttgcagcagggctgc
tggctggtggtccccttgtggggatcctttgctcttactctaagatagtttcctccatacgtgcaatctcatcagct
caggggaagtacaaggcattttccacctgtgcatcacacctctcagttgtctctttattttgttgtacgggcctagg
tgtgtaccttacttctgctgcaacccacaactcacacacaagtgcaacagcctcagtgatgtacactgtggccaccc
ccatgctgaacccctttatctacagtctgaggaataaagacataaagagggctctgaaaatgtccttcagaggaaag
caataa
Protein
SEQ ID NO: 25
MEPENDTGISEFVLLGLSEEPELQPFLFGLFLSMYLVTVLGNLLIILATISDSHLHTPMYFFLSNLSFADICFISTT
IPKMLINIQTQSRVITYAGCITQMCFFVLFGGLDSLLLAVMAYDRFVAICHPLHYTVIMNPRLCGLLVLASWMIAAL
NSLSQSLMVLWLSFCTDLEIPHFFCELNQVIHLACSDTFLNDMGMYFAAGLLAGGPLVGILCSYSKIVSSIRAISSA
QGKYKAFSTCASHLSVVSLFCCTGLGVYLTSAATHNSHTSATASVMYTVATPMLNPFIYSLRNKDIKRALKMSFRGK
Q
OR10J5
DNA
SEQ ID NO: 26
atgaagagaaagaacttcacagaagtgtcagaattcattttcttgggattttctagctttggaaagcatcagataac
cctctttgtggttttcctaactgtctacattttaactctggttgctaacatcatcattgtgactatcatctgcattg
accatcatctccacactcccatgtatttcttcctaagcatgctggctagttcagagacggtgtacacactggtcatt
gtgccacgaatgcttttgagcctcatttttcataaccaacctatctccttggcaggctgtgctacacaaatgttctt
ttttgttatcttggccactaataattgcttcctgcttactgcaatggggtatgaccgctatgtggccatctgcagac
ccctgagatacactgtcatcatgagcaagggactatgtgcccagctggtgtgtgggtcctttggcattggtctgact
atggcagttctccatgtgacagccatgttcaatttgccgttctgtggcacagtggtagaccacttcttttgtgacat
ttacccagtcatgaaactttcttgcattgataccactatcaatgagataataaattatggtgtaagttcatttgtga
tttttgtgcccataggcctgatatttatctcctatgtccttgtcatctcttccatccttcaaattgcctcagctgag
ggccggaagaagacctttgccacctgtgtctcccacctcactgtggttattgtccactgtggctgtgcctccattgc
ctacctcaagccgaagtcagaaagttcaatagaaaaagaccttgttctctcagtgacgtacaccatcatcactccct
tgctgaaccctgttgtttacagtctgagaaacaaggaggtaaaggatgccctatgcagagttgtgggcagaaatatt
tcttaa
Protein
SEQ ID NO: 27
MKRKNFTEVSEFIFLGFSSFGKHQITLFVVFLTVYILTLVANIIIVTIICIDHHLHTPMYFFLSMLASSETVYTLVI
VPRMLLSLIFHNQPISLAGCATQMFFFVILATNNCFLLTAMGYDRYVAICRPLRYTVIMSKGLCAQLVCGSFGIGLT
MAVLHVTAMFNLPFCGTVVDHFFCDIYPVMKLSCIDTTINEIINYGVSSFVIFVPIGLIFISYVLVISSILQIASAE
GRKKTFATCVSHLTVVIVHCGCASIAYLKPKSESSIEKDLVLSVTYTIITPLLNPVVYSLRNKEVKDALCRVVGRNI
S
OR1C1
DNA
SEQ ID NO: 28
atggaaaaaagaaatctaacagttgtcagggaattcgtccttctgggacttcctagctcagcagagcagcagcacct
cctgtctgtgctctttctctgtatgtatttagccaccaccttggggaacatgctcatcattgcgacgattggctttg
actctcacctccattcccctatgtacttcttccttagtaacttggcctttgttgacatctgctttacgtcgactaca
gtcccccaaatggtagtgaatatcttgactggcaccaagactatctcttttgcaggctgcctcacccagctcttctt
cttcgtttcttttgtgaatatggacagcctccttctgtgtgtgatggcgtatgatagatatgtggcgatttgccacc
ccttacattacaccgccagaatgaacctgtgcctttgtgtccagctagtggctggactgtggcttgttacttacctc
cacgccctcctgcatactgtcctaatagcacagctgtccttctgtgcctccaatatcatccatcatttcttctgtga
tctcaatcctctcctgcagctctcttgctctgacgtctccttcaatgtaatgatcatttttgcagtaggaggtctat
tggctctcacgccccttgtctgtatcctcgtatcttatggacttatcttctccactgttctgaagatcacctctact
cagggcaagcagagagctgtttccacctgcagctgccacctgtcagtggtggtgttgttttacggcacagccatcgc
cgtctatttcagcccttcatccccccatatgcctgagagcgacactctgtcaaccatcatgtattcaatggtggctc
cgatgctgaatcctttcatctataccctaaggaacagggatatgaagaggggacttcagaaaatgcttctcaagtgc
acagtctttcagcagcaataa
Protein
SEQ ID NO: 29
MEKRNLTVVREFVLLGLPSSAEQQHLLSVLFLCMYLATTLGNMLIIATIGFDSHLHSPMYFFLSNLAFVDICFTSTT
VPQMVVNILTGTKTISFAGCLTQLFFFVSFVNMDSLLLCVMAYDRYVAICHPLHYTARMNLCLCVQLVAGLWLVTYL
HALLHTVLIAQLSFCASNIIHHFFCDLNPLLQLSCSDVSFNVMIIFAVGGLLALTPLVCILVSYGLIFSTVLKITST
QGKQRAVSTCSCHLSVVVLFYGTAIAVYFSPSSPHMPESDTLSTIMYSMVAPMLNPFIYTLRNRDMKRGLQKMLLKC
TVFQQQ
OR5B12
DNA
SEQ ID NO: 30
atggagaacaacacagaggtgactgaattcatccttgtggggttaactgatgacccagaactgcagatcccactctt
catagtcttccttttcatctacctcatcactctggttgggaacctggggatgattgaattgattctactggactcct
gtctccacacccccatgtacttcttcctcagtaacctctccctggtggactttggttattcctcagctgtcactccc
aaggtgatggtggggtttctcacaggagacaaattcatattatataatgcttgtgccacacaattcttcttctttgt
agcctttatcactgcagaaagtttcctcctggcatcaatggcctatgaccgctatgcagcattgtgtaaacccctgc
attacaccaccaccatgacaacaaatgtatgtgcttgcctggccataggctcctacatctgtggtttcctgaatgca
tccattcatactgggaacactttcaggctctccttctgtagatccaatgtagttgaacactttttctgtgatgctcc
tcctctcttgactctctcatgttcagacaactacatcagtgagatggttattttttttgtggtgggattcaatgacc
tcttttctatcctggtaatcttgatctcctacttatttatatttatcaccatcatgaagatgcgctcacctgaagga
cgccagaaggccttttctacttgtgcttcccaccttactgcagtttccatcttttatgggacaggaatctttatgta
cttacgacctaactccagccatttcatgggcacagacaaaatggcatctgtgttctatgccatagtcattcccatgt
tgaatccactggtctacagcctgaggaacaaagaggttaagagtgcctttaaaaagactgtagggaaggcaaaggcc
tctataggattcatattttaa
Protein
SEQ ID NO: 31
MENNTEVTEFILVGLTDDPELQIPLFIVFLFIYLITLVGNLGMIELILLDSCLHTPMYFFLSNLSLVDFGYSSAVTP
KVMVGFLTGDKFILYNACATQFFFFVAFITAESFLLASMAYDRYAALCKPLHYTTTMTTNVCACLAIGSYICGFLNA
SIHTGNTFRLSFCRSNVVEHFFCDAPPLLTLSCSDNYISEMVIFFVVGFNDLFSILVILISYLFIFITIMKMRSPEG
RQKAFSTCASHLTAVSIFYGTGIFMYLRPNSSHFMGTDKMASVFYAIVIPMLNPLVYSLRNKEVKSAFKKTVGKAKA
SIGFIF

The following examples are illustrative only and are not meant to limit the scope of invention as set forth in the Summary, Description or in the Claims.

### EXAMPLES

Tonality descriptions in the Examples below were provided by one or more perfumer.

### Example 1: Olfactory Quality Encoding by the Peripheral Olfactory System

The data presented below is summarized in FIGS. 1 and 2 to conceptually capture how olfactory tonalities are encoded at the periphery of the olfactory system. In FIG. 1, a fragrance wheel representing perfumery notes and tonalities, and the relationships among the corresponding olfactory groups is shown. Volatile compounds exhibiting particular olfactory tonalities on the fragrance wheel have been mapped by the receptors they activate. Molecules sharing a tonality activate the same OR, irrespective of chemical similarity. Molecules that share several olfactory tonalities consequently also co-activate the same corresponding ORs. For example molecules 1, 2 and 3 each activate two characterized receptors and exhibit the corresponding 2 olfactory tonalities (as shown in FIG. 1).

This is further illustrated in FIG. 2. It shows the receptive field of three receptors—OR5AU1 (SEQ ID NO. 15), OR8D1 (SEQ ID NO. 17) and OR8B3 (SEQ ID NO. 13)—each linked to a distinct olfactory tonality—lactonic coconut, fenugreek or coumarinic, respectively, wherein fenugreek is captured by the semantically related descriptors fenugreek, maple and celery, and coumarinic by coumarin, tonka, hay. Molecules that exhibit only one of these tonalities activate only one receptor, correspondingly. Molecules that exhibit a combination of these descriptors turn out to activate the combination of corresponding receptors. The compounds at the intersection of two or more OR receptive fields will elicit the two or more corresponding tonalities (FIG. 2). As a result, the overall receptor activation profile (i.e. which set of receptors are activated) correlates directly with the sensory attributes of a given compound. Table 1 lists the tonalities of a subset of compounds next to the characterized receptors shown in FIG. 2. Additional descriptions not represented by OR activity suggest that additional ORs remain to be deorphanized and characterized. Of note, the data presented in this example is not intended to be exhaustive. There are many more olfactory tonalities than the ones represented in this fragrance wheel and there are many more OR-odorant pairs. The predictive aspect of OR screening, however, becomes clear in the light of the summarized results.

TABLE 1
Compounds activating multiple ORs share corresponding tonalities

| | Compound | CAS | IUPAC | | Organoleptic | Active OR/Linked |
|---|---|---|---|---|---|---|
| | Name | No | Name | Smiles | Descriptors | Tonality |
| 1. | Gamma | 000706-14-9 | 5-hexyloxolan-2-one | O═C1CCC(CCCCCC)O1 | fresh, oily, waxy, | OR5AU1/ |
| | Decalactone | | | | peach, coconut, | Lactonic |
| | | | | | buttery, sweet | Coconut |
| 2. | Delta | 032764-98-0 | 6-pent-3-enyloxan-2-one | O═C1CCCC(CC/C═C/ | oily, fruity, coconut, | OR5AU1/ |
| | Jasmolactone | | | C)O1.O═C2CCCC(CC/ | floral, petal absolute, | Lactonic |
| | | | | C═C\C)O2 | jasmin, tuberose, | Coconut |
| | | | | | peach, apricot | |
| 4. | Octalactone | 000698-76-0 | 6-propyloxan-2-one | O═C1CCCC(CCC)O1 | celery, tonka, butter, | OR5AU1/ |
| | Delta | | | | coconut | Lactonic |
| | | | | | | Coconut |
| | | | | | | OR8D1/ |
| | | | | | | Fenugreek |
| | | | | | | OR8B3/ |
| | | | | | | Coumarinic |
| 5. | Whiskey | 39638-67-0 | 5-butyl-4- | CCCCC(O1)C(C)CC1═O | tonka, coumarin, | OR5AU1/ |
| | Lactone K | | methyloxolan-2-one | | coconut, toasted, | Lactonic |
| | | | | | nutty, celery, burnt | Coconut |
| | | | | | | OR8D1/ |
| | | | | | | Fenugreek |
| | | | | | | OR8B3/ |
| | | | | | | Coumarinic |
| 6. | Gamma | 000104-50-7 | 5-butyloxolan-2-one | O═C1CCC(CCCC)O1 | sweet, coconut, waxy, | OR5AU1/ |
| | Octalactone | | | | creamy, tonka, dairy, | Lactonic |
| | | | | | fatty | Coconut |
| | | | | | | OR8B3/ |
| | | | | | | Coumarinic |
| 7. | Nonalactone | 003301-94-8 | 6-butyloxan-2-one | O═C1CCCC(CCCC)O1 | coconut, creamy, | OR5AU1/ |
| | Delta | | | | sweet, milky, | Lactonic |
| | | | | | coumarin | Coconut |
| | | | | | | OR8B3/ |
| | | | | | | Coumarinic |
| 10. | Sotolone | 028664-35-9 | 4-hydroxy-2,3- | CC(C(C)O1)═C(O)C1═O | celery, fenugreek, | OR8D1/ |
| | | | dimethyl-2~{H}- | | very powerful | Fenugreek |
| | | | furan-5-one | | | |
| 11. | Maple | 000698-10-2 | 2-ethyl-4-hydroxy-3- | CC(C(CC)O1)═C(O)C1═O | sweet, fruity, | OR8D1/ |
| | Furanone | | methyl-2~{H}-furan- | | caramellic, maple, | Fenugreek |
| | | | 5-one | | fenugreek, brown | |
| | | | | | sugar, nutty, chicory, | |
| | | | | | praline, butterscotch | |
| 13. | Tricyclone | 068433-81-8 | (1~{R},7~{R},9~{S})- | [H][C@@]1(CC2)C[C | hay, honey, tobacco, | OR8B3/ |
| | | | 10,10-dimethyltricyclo[7.1.1.0\hat{ }{ | @@H](C3)C(C)(C)[C | very natural | Coumarinic |
| | | | 2,7}]undec-2-en-4-one | @@H]3C1═CC2═O | | |
| 14. | Florex | 69486-14-2 | 9-ethylidene-3- | O═C1OC2C(CC1)C3C | sweet, spicy, balsam, | OR8B3/ |
| | | | oxatricyclo[6.2.1.0\hat{ }{ | C2CC3═CC.O═C4CCC | tonka, woody, | Coumarinic |
| | | | 2,7}]undecan-4- | (C(C5)C6)C(C6C5═CC)O4 | tobacco, green | |
| | | | one; 10-ethylidene-3- | | | |
| | | | oxatricyclo[6.2.1.0\hat{ }{ | | | |
| | | | 2,7}]undecan-4-one | | | |

### Example 2: OR11A1 Activity Captures a Single Common Organoleptic Tonality: Earthy

The molecular receptive range of the human odorant receptor OR11A1 (SEQ ID NO. 11) was tested by performing a large scale screening with a chemically and organoleptically diverse volatile compound library comprising approximately 800 compounds. Using a cell-based assay, OR11A1 was tested in an HEK293T cell line wherein the endogenous RTP1 gene has been activated and the odorant receptor chaperone was expressed (WO2016/201153 A1). Cells were co-transfected with the Flag-Rho-tagged receptor and the canonical olfactory G-protein G_{olf}, and exposed to a single concentration of each test compound, tested individually. Receptor activity was detected by measuring the cAMP increase in the cytosol using an HTRF (Homogenous Time-Resolved Fluorescence unit) based kit (CisBio, cAMP dynamic 2 kit, 62AM4PEJ).

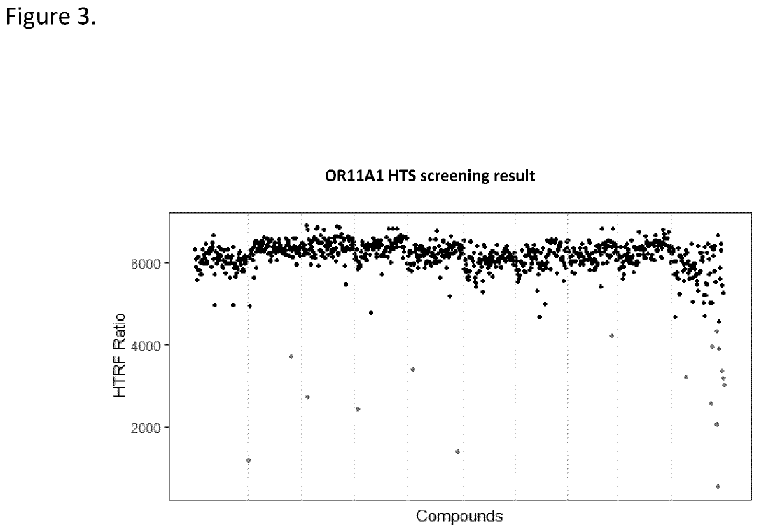

First, 300 mM stock solutions for 798 test compounds were made in pure DMSO. The stock solutions for each compound were further diluted to a final test concentration of 300 μM. The final concentration of DMSO was 0.1%, and had no visible effect on the cells. Each compound was presented to a cell line expressing the OR11A1 olfactory receptor. The quality of this high-throughput screening (HTS) process was determined and the window variability and signal reliability were assessed by calculating the Z′ value of each plate (the average Z′ value per plate was 0.72±0.12, well within the required quality standards between 0.5 and 1). The result of this single concentration activation screen is shown in FIG. 3, where hits are highlighted in light grey dots.

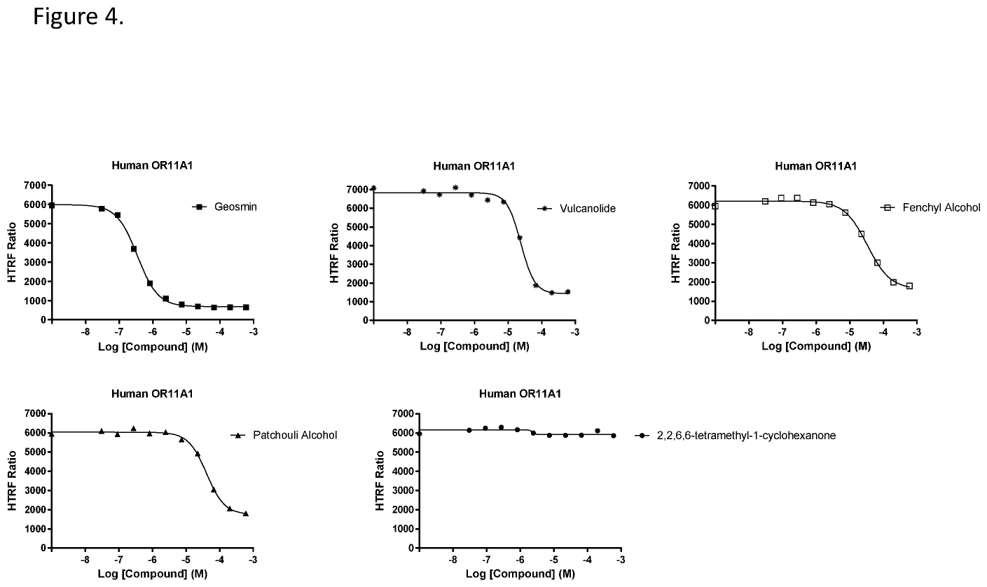

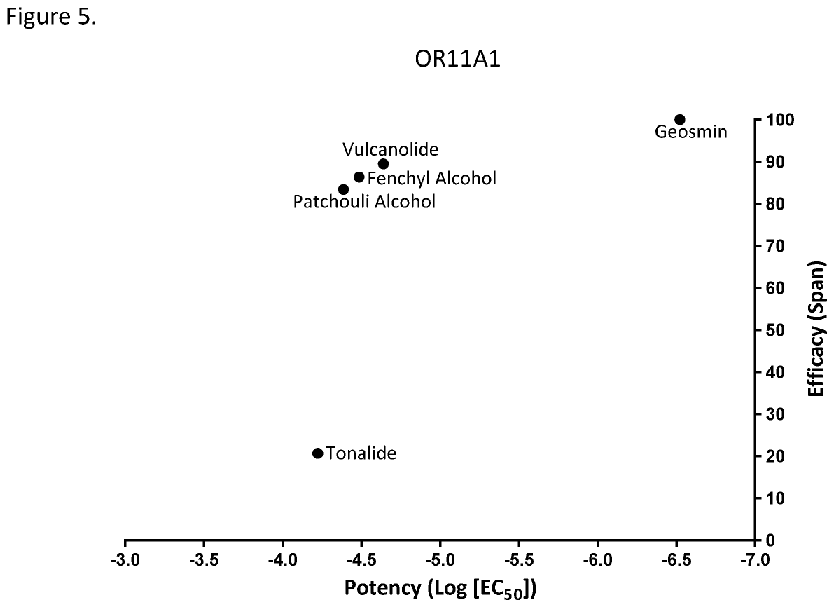

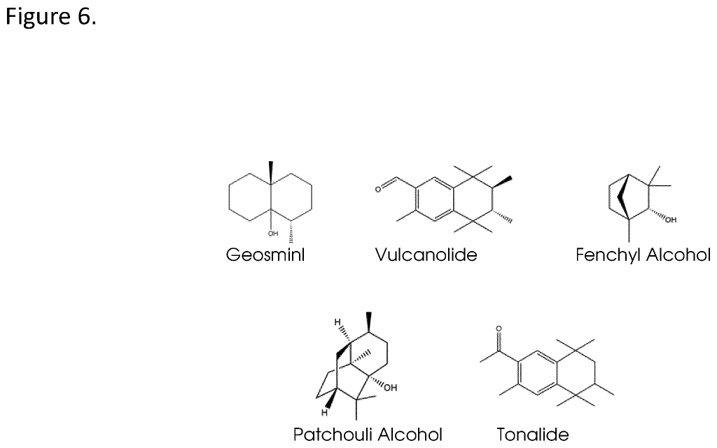

In a subsequent step using the same cell-based assay described above, the candidate hits were confirmed to be true agonists (activators) by performing dose-response experiments as shown in FIG. 4 for a subset of the best hits. A negative compound from the initial screening step (2,2,6,6-tetramethyl-1-cyclohexanone) was used a negative control for response specificity and did not yield any significant dose-response. Both sensitivity (potency) and activation strength (efficacy) were calculated from the curves, as a measure of EC₅₀ (the concentration required to reach half-maximal activation level) and Span (the assay window span between baseline activity level and activation saturation plateau), respectively. These values were used to represent the molecular receptive range of OR11A1 according to the potency (X axis) and efficacy (Y axis) in FIG. 5. The corresponding chemical structures are indicated in FIG. 6. A partial agonist, Tonalide, is also listed. The five agonists cover distinct perfumery tonalities: woody (Patchouli Alcohol), musky (Vulcanolide, Tonalide), and camphorous (Fenchyl Alcohol), Table 2. Taken together, the data depict the diversity of both the organoleptic tonalities and chemical structures of compounds capable of activating OR11A1, congruent with results described in WO2016/201152 A1 (musk) and EP2832347A1.(patchouli). The OR11A1 activity however was not associated with the tonalities of musk (WO2016/201152 A1) or patchouli (EP2832347A1) specifically but rather the earthy facet thereof. In fact, the single commonality between these compounds was the earthy tonality, which is semantically captured by the following descriptors: earthy, humus and earthy-moss. These compounds are neither all musks nor patchouli but they all are earthy. In contrast, Tonalide, a musk structurally related to Vulcanolide but only a weak (partial) agonist of OR11A1, did not deliver a strong earthy tonality in spite of chemical and overall organoleptic similarity to Vulcanolide (i.e. musky, powdery), as shown in Table 2.

TABLE 2
Compounds activating OR11A1 share the olfactory earthy tonality.

| | Compound | CAS | IUPAC | | Organoleptic | OR11A1 |
|---|---|---|---|---|---|---|
| | Name | No | Name | Smiles | Descriptors | % Efficacy |
| 1. | Geosmin | 19700-21-1 | (4~{S},8~{a}~{R})-4,8~{a}- | OC1(CCCC2)[C@@]2(C) | humus, camphor, | 100% |
| | | | dimethyl-1,2,3,4,5,6,7,8- | CCC[C@@H]1C | earthy-moss | (full |
| | | | octahydronaphthalen-4~{a}-ol | | | agonist) |
| 2. | Vulcanolide | 127459-79-4 | (6~{R},7~{R})-3,5,5,6,7,8,8- | C[C@H](C(C1═CC(C)═C( | musk, powdery, | 89% |
| | | | heptamethyl-6,7- | C═O)C═C21)(C)C)[C@H]( | earthy-moss | |
| | | | dihydronaphthalene-2- | C)C2(C)C | | |
| | | | carbaldehyde | | | |
| 3. | Fenchyl | 14575-74-7 | (1~{R},2~{R},4~{S})-1,3,3- | C[C@]12CC[C@@H](C2) | camphor, earthy- | 86% |
| | Alcohol | | trimethylbicyclo[2.2.1]heptan-2-ol | C(C)(C)[C@@H]1O | moss, floral | |
| 4. | Patchouli | 005986-55-0 | (1~{S},3~{S},6~{R},7~{R},8 | C[C@@]12CC[C@@]3([ | earthy, camphor, | 83% |
| | Alcohol | | ~{S})-2,2,6,8- | H])C(C)(C)[C@]1(O)CC[ | powdery | |
| | | | tetramethyltricyclo[5.3.1,0\hat{ }{3, | C@H](C)[C@]2([H])C3 | | |
| | | | 8}]undecan-3-ol | | | |
| 5. | Tonalide | 21145-77-7 | 1-(3,5,5,6,8,8-hexamethyl-6,7- | CC1CC(C2═C(C1(C)C)C═ | sweet, amber, | 21% |
| | | | dihydronaphthalen-2-yl)ethanone | C(C(═C2)C(═O)C)C)(C)C | fruity, musk, | (partial |
| | | | | | powdery | agonist) |

### Example 3: Odorant Receptors Encode Specific Organoleptic Tonalities

Following the same approach described in Example 2, a systematic screening of multiple Lucy-Flag-Rho tagged human ORs was applied in search of the link between the receptor activity and the particular olfactory tonality they encode. The analysis of the molecular receptive ranges of ORs shows that agonists may vary in their physicochemical properties (e.g. molecular weight, volatility, functional group, 3-dimensional structure, etc.) and in their overall organoleptic tonalities, yet a common particular olfactory tonality can be identified in all cases. As a result, the following receptors have been attributed to specific olfactory tonalities and are considered fundamental in eliciting the perception of the tonality.

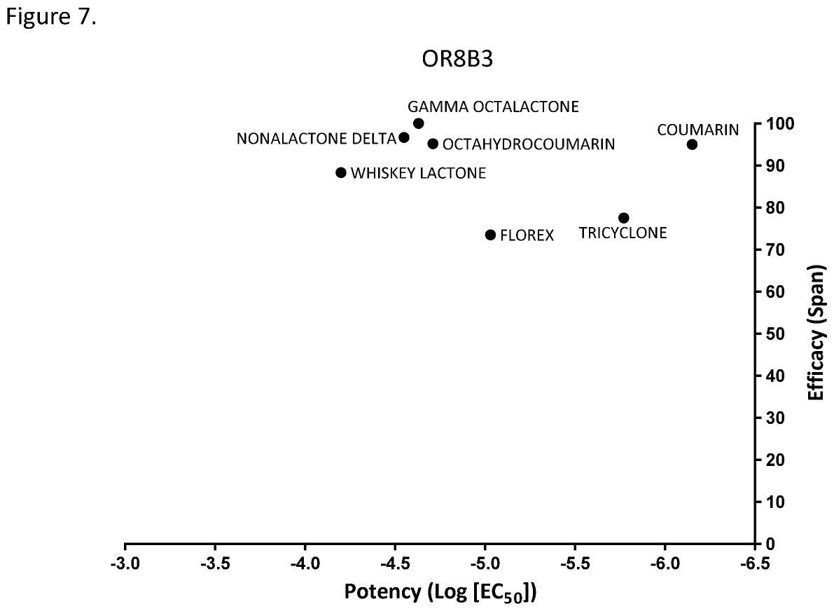

OR8B3, SEQ ID NO. 13, was activated by compounds generating a coumarinic tonality (FIG. 7, Table 3).

TABLE 3
Compounds activating OR8B3 share the olfactory coumarinic tonality.

| | Compound | CAS | IUPAC | | Organoleptic | OR8B3 |
|---|---|---|---|---|---|---|
| | Name | No | Name | Smiles | Descriptors | % Efficacy |
| 1. | Coumarin | 000091-64-5 | chromen-2-one | O═C1OC2═CC═CC═C2C═ | sweet, hay, tonka, | 95% |
| | | | | Cl | new mown hay | |
| 2. | Gamma | 000104-50-7 | 5-butyloxolan-2-one | O═C1CCC(CCCC)O1 | sweet, coconut, | 100% |
| | octalactone | | | | waxy, creamy, | (full |
| | | | | | tonka, dairy, fatty | agonist) |
| 3. | Nonalactone | 003301-94-8 | 6-butyloxan-2-one | O═C1CCCC(CCCC)O1 | coconut, creamy, | 97% |
| | delta | | | | sweet, milky, | |
| | | | | | coumarin | |
| 4. | Octahydrocoumarine | 004430-31-3 | 3,4,4~{a},5,6,7,8,8~{a}- | O═C1CCC2C(O1)CCCC2 | sweet, herbal, | 95% |
| | | | octahydrochromen-2-one | | coumarin, tonka, | |
| | | | | | woody | |
| 5. | Whiskey | 39638-67-0 | 5-butyl-4-methyloxolan-2-one | CCCCC(O1)C(C)CC1═O | coconut, citrus, | 88% |
| | lactone | | | | sweet, bitter, | |
| | | | | | coumarin | |
| 6. | Tricyclone | 068433-81-8 | (1~{R},7~{R},9~{S})-10,10- | [H][C@@]1(CC2)C[C@@ | powdery, tonka | 77% |
| | | | dimethyltricyclo[7.1.1.0\hat{ }{2,7 | H](C3)C(C)(C)[C@@H]3 | | |
| | | | }]undec-2-en-4-one | C1═CC2═O | | |
| 7. | Florex | 249630-78-2 | 9-ethylidene-3- | O═C1OC2C(CC1)C3CC2C | rhubarb, lactonic, | 74% |
| | | | oxatricyclo[6.2.1.0\hat{ }{2,7}]und | C3═CC.O═C4CCC(C(C5) | coumarin, tonka | |
| | | | ecan-4-one; 10-ethylidene-3- | C6)C(C6C5═CC)O4 | | |
| | | | oxatricyclo[6.2.1.0\hat{ }{2,7}]und | | | |
| | | | ecan-4-one | | | |

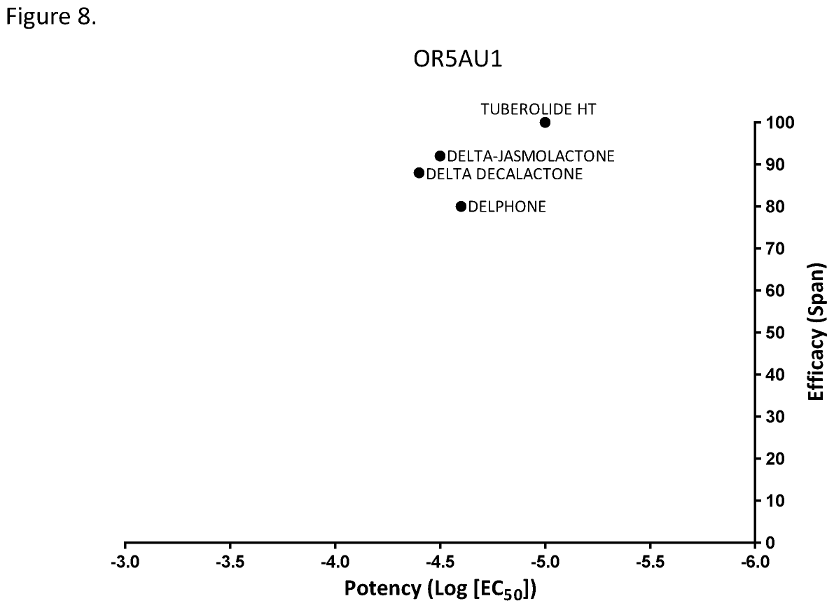

OR5AU1, SEQ ID NO. 15, was activated by compounds generating a lactonic coconut tonality (FIG. 8, Table 4).

TABLE 4
Compounds activating OR5AU1 share the olfactory lactonic coconut tonality.

| | | | | | | OR5AU1 |
|---|---|---|---|---|---|---|
| | Compound | CAS | IUPAC | | Organoleptic | % |
| | Name | No | Name | Smiles | Descriptors | Efficacy |
| 1. | Tuberolide | 089631-04-9 | (4~{R},5~{R})-4-methyl-5- | C[C@H]([C@@H](CCCC | waxy, lactonic, | 100% |
| | Ht | | pentyloxolan-2-one | C)O1)CC1═O | metallic, tuberose, plum | (full |
| | | | | | | agonist) |
| 2. | Delta- | 032764-98-0 | 6-pent-3-enyloxan-2-one | O═C1CCCC(CC/C═C/C)O | oily, fruity, coconut, | 92% |
| | Jasmolactone | | | 1.O═C2CCCC(CC/C═C\C) | floral, petal absolute, | |
| | | | | O2 | jasmin, tuberose, peach, | |
| | | | | | apricot | |
| 3. | Delta | 000705-86-2 | 6-pentyloxan-2-one | O═C1CCCC(CCCCC)O1 | fresh, sweet, oily, | 88% |
| | decalactone | | | | coconut, fruity, peach, | |
| | | | | | creamy, dairy | |
| 4. | Delphone | 004819-67-4 | 2-pentylcyclopentan-1 -one | O═C1CCCC1CCCCC | coconut, jasmin, oily, | 80% |
| | | | | | rose, fem, green, herbal | |

OR8D1, SEQ ID NO. 17, was activated by compounds generating a fenugreek tonality

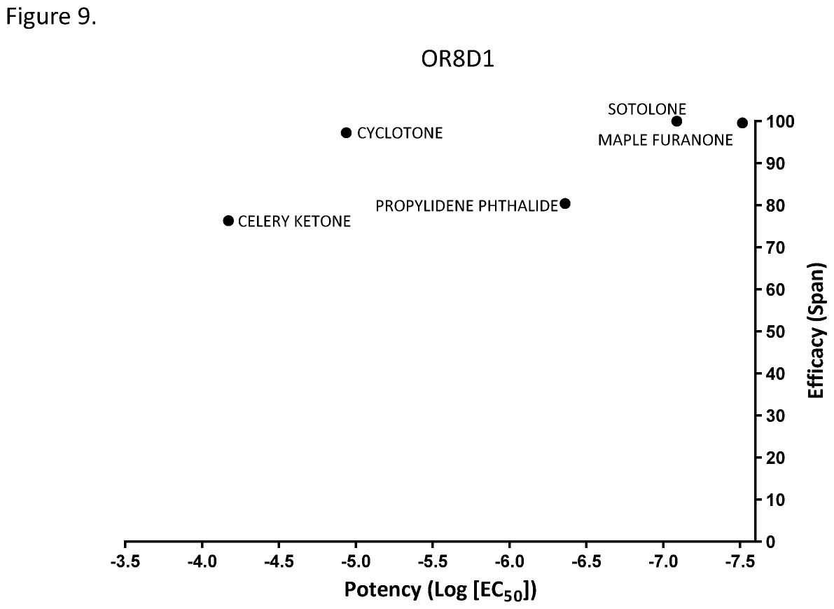

(FIG. 9, Table 5).

TABLE 5
Compounds activating OR8D1 share the olfactory fenugreek tonality.

| | Compound | CAS | IUPAC | | Organoleptic | OR8D1 |
|---|---|---|---|---|---|---|
| | Name | No | Name | Smiles | Descriptors | % Efficacy |
| 1. | Maple | 000698-10-2 | 2-ethyl-4-hydroxy-3-methyl- | CC(C(CC)O1)═C(O)C1═O | sweet, fruity, | 100% |
| | furanone | | 2~{H}-furan-5-one | | caramellic, maple, | (full |
| | | | | | fenugreek, brown | agonist) |
| | | | | | sugar, nutty, chicory, | |
| | | | | | praline, butterscotch | |
| 2. | Sotolone | 028664-35-9 | 4-hydroxy-2,3-dimethyl- | CC(C(C)O1)═C(O)C1═O | extremely sweet, | 100% |
| | | | 2~{H}-furan-5-one | | strong, caramel, maple, | (full |
| | | | | | burnt sugar, coffee | agonist) |
| 3. | Propylidene | 94704-09-9 | 3-propylidene-2-benzofuran- | O═C(O/1)C2═C(C1═C\CC) | celery, maple, | 80% |
| | phthalide | | 1-one | C═CC═C2.O═C(O/3)C4═ | fenugreek | |
| | | | | C(C3═C/CC)C═CC═C4 | | |
| 4. | Cyclotone | 000080-71-7 | 2-hydroxy-3- | CC(CC1)═C(O)C1═O | caramel, maple syrup, | 97% |
| | | | methylcyclopent-2-en-1-one | | sweet, maple, sugar, | |
| | | | | | coffee, woody | |
| 5. | Celery | 003720-16-9 | 3-methyl-5-propylcyclohex- | O═C1C═C(C)CC(CCC)C1 | fresh, celery, green, | 76% |
| | ketone | | 2-en-1-one | | cumin | |

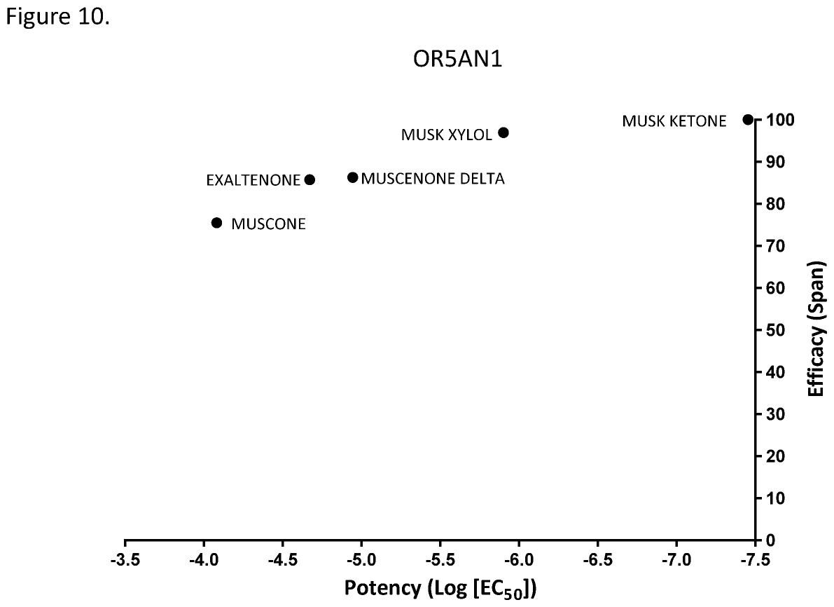

OR5AN1, SEQ ID NO. 19, was activated by structurally distinct molecules such as nitromusks and macrocyclic ketones generating a powdery musk tonality, commonly used in perfumery (FIG. 10, Table 6).

TABLE 6
Compounds activating OR5AN1 share the olfactory powdery musk tonality.

| | Compound | CAS | IUPAC | | Organoleptic | OR5AN1 |
|---|---|---|---|---|---|---|
| | Name | No | Name | Smiles | Descriptors | % Efficacy |
| 1. | Musk ketone | 81-14-1 | 1-(4-~{tert}-butyl-2,6- | CC(C([N+]([O-])═ | fatty, musk, soapy, | 100% |
| | | | dimethyl-3,5- | O)═C1C(C)(C)C)═C(C( | dry, powdery | (full |
| | | | dinitrophenyl)ethanone | C)═O)C(C)═C1[N+] | | agonist) |
| | | | | ([O-])═O | | |
| 2. | Musk xylol | 81-14-1 | 1-~{tert}-butyl-3,5-dimethyl- | CC(C)(C)C(C([N+] | fatty, dry, sweet, | 97% |
| | | | 2,4,6-trinitrobenzene | ([O-])═O)═C(C)C([N+] | soapy, musk | |
| | | | | ([O-])═O)═C1C)═C1[N+] | | |
| | | | | ([O-])═O | | |
| 3. | Muscenone | 464207-51-0 | 3-methylcyclopentadec-4-en- | CC(C1)/C═C/CCCCCCCC | musk, ketone, | 86% |
| | delta | | 1-one; 3-methylcyclopentadec- | CCC1═O.CC(C2)C/C═C/C | animal, soapy | |
| | | | 5-en-1-one | CCCCCCCCC2═O.CC(C3) | | |
| | | | | C/C═C\CCCCCCCCCC3 | | |
| | | | | ═O | | |
| 4. | Exaltenone | 014595-54-1 | cyclopentadec-4-en-1-one | O═C1CCCCCCCCCC/C═ | musk, animal, | 86% |
| | | | | C\CC1 | natural | |
| 5. | Muscone | 10403-00-6 | (R)-3- | CC1CCCCCCCCCCCCC | sweet, musk, animal, | 75% |
| | | | methylcyclopentadecanone | (═O)C1 | powdery, fatty, | |
| | | | | | natural | |

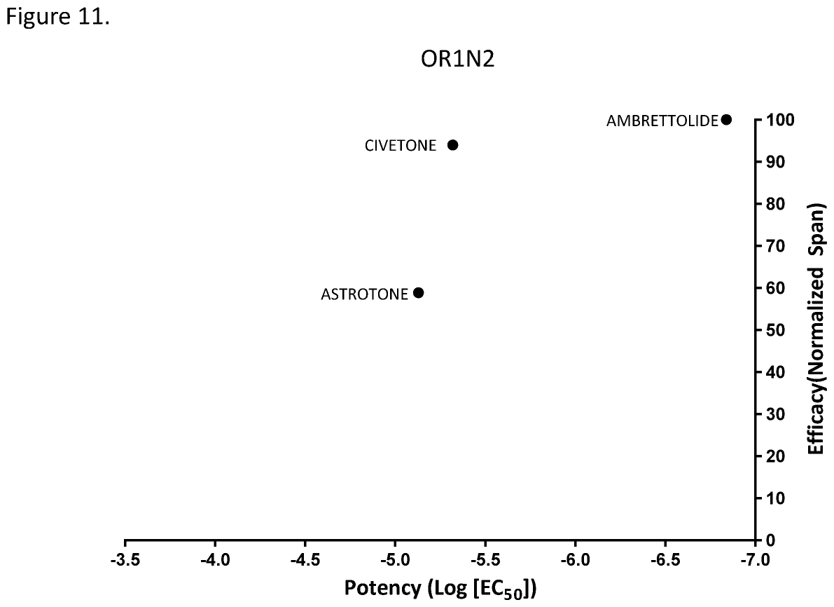

OR1N2, SEQ ID NO. 21, was activated by large macrocylic compounds (ketones and lactones), generating an animalic musk tonality, commonly used in perfumery (FIG. 11, Table 7).

TABLE 7
Compounds activating OR1N2 share the olfactory animalic musk tonality.

| | Compound | CAS | IUPAC | | Organoleptic | OR1N2 |
|---|---|---|---|---|---|---|
| | Name | No | Name | Smiles | Descriptors | % Efficacy |
| 1. | Ambrettolide | 028645-51-4 | 1-oxacycloheptadec-10-en-2- | O═C1CCCCCCC/C═C/CC | sweet, musk, | 100% |
| | | | one | CCCCO1 | ambrette, fruity, | (full |
| | | | | | waxy, fatty | agonist) |
| 2. | Civetone | 542-46-1 | cycloheptadec-9-en-1-one | O═C1CCCCCCCC═CCCC | clean, musk, dry, | 94% |
| | | | | CCCC1 | animal, sweet | |
| 3. | Astrotone | 000105-95-3 | 1,4-dioxacycloheptadecane- | O═C(OCCO1)CCCCCCC | powdery, sweet, | 59% |
| | | | 5,17-dione | CCCCC1═O | floral, ambrette, | |
| | | | | | musk, woody | |

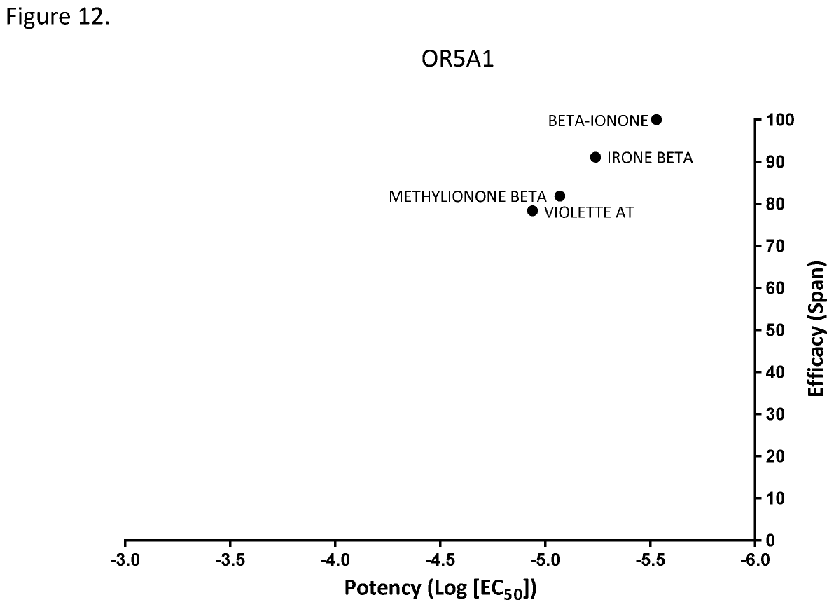

OR5A1, SEQ ID NO. 23, was activated by compounds generating a violet tonality (FIG. 12, Table 8).

TABLE 8
Compounds activating OR5A1 share the olfactory violet tonality.

| | Compound | CAS | IUPAC | | Organoleptic | OR5A1 |
|---|---|---|---|---|---|---|
| | Name | No | Name | Smiles | Descriptors | % Efficacy |
| 1. | Beta-ionone | 79-77-6 | 4-(2,6,6-trimethylcyclohexen- | CC1(C)C(/C═C/C(C)═O)═ | dry, powdery, floral, | 100% |
| | | | 1-yl)but-3-en-2-one | C(C)CCC1 | woody, orris, sweet, | (full |
| | | | | | fruity, berry, tropical, | agonist) |
| | | | | | beeswax, violet | |
| 2. | Irone beta | 000079-70-9 | 4-(2,5,6,6- | CC1(C)C(/C═C/C(C)═O)═ | fresh, sweet, violet, | 91% |
| | | | tetramethylcyclohexen-1- | C(C)CCC1C | berry, woody, | |
| | | | yl)but-3-en-2-one | | powdery | |
| 3. | Methylionone | 063429-28-7 | 1-(2,6,6-trimethylcyclohexen- | CC1(C)C(/C═C/C(CC)═O)═ | floral, orris, woody, | 82% |
| | beta | | 1-yl)pent-1-en-3-one | C(C)CCC1 | powdery, violet, | |
| | | | | | tropical, tobacco, | |
| | | | | | creamy | |
| 4. | Violette AT | 127-41-3 | 4-(2,6,6-trimethylcyclohexen- | CC1(C)C(/C═C/C(C)═O)═ | sweet, woody, floral, | 78% |
| | | | 1-yl)but-3-en-2-one; 4-(2,6,6- | C(C)CCC1.CC2(C)C(/C═C/ | violet, orris, | |
| | | | trimethylcyclohex-2-en-1- | C(C)═O)C(C)═CCC2 | tropical, fruity | |
| | | | yl)but-3-en-2-one | | | |

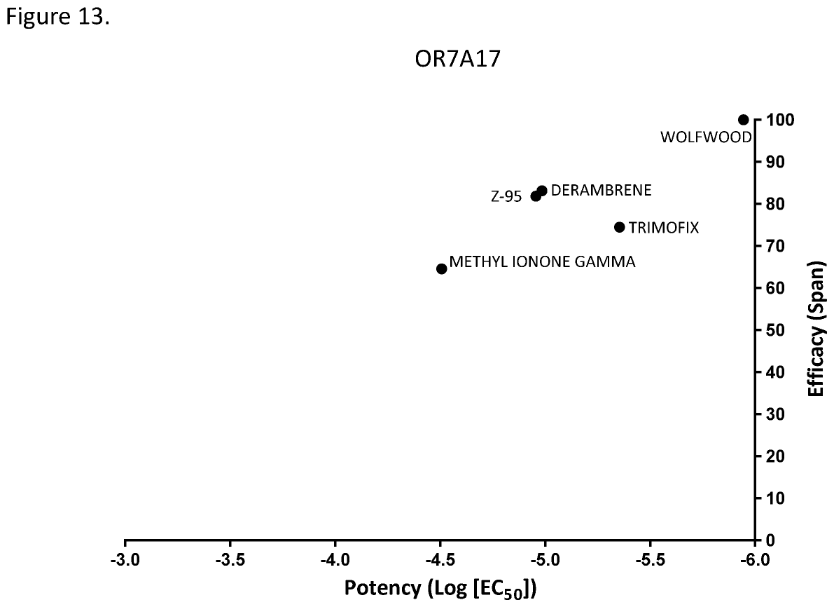

OR7A17, SEQ ID NO. 25, was activated by compounds generating a blonde wood (also described as creamy wood) tonality (FIG. 13, Table 9).

TABLE 9
Compounds activating OR7A17 share the olfactory blonde wood tonality.

| | Compound | CAS | IUPAC | | Organoleptic | OR7A17 |
|---|---|---|---|---|---|---|
| | Name | No | Name | Smiles | Descriptors | % Efficacy |
| 1. | Wolfwood | 133636-82-5 | (1~{S},2~{R},3~{R},5~{R})- | CC1(C)[C@H]2C[C@@H] | woody, nicely | 100% |
| | | | 2,6,6- | 1C[C@](C═CC3═O)(CC3) | woody, | (full |
| | | | trimethylspiro[bicyclo[3.1.1]h | [C@H]2C | methylionone | agonists) |
| | | | eptane-3,4′-cyclohex-2-ene]- | | | |
| | | | 1′-one | | | |
| 2. | Derambrene | 145206-68-4 | 1-(2,3,8,8-tetramethyl- | CC1(C(C)═O)CC(C(CCC2 | woody, dry, ambergris, | 83% |
| | | | 1,3,4,6,7,8~{a}- | )(C)C)═C2CC1C.CC3(C)C | cedar, woody, old, | |
| | | | hexahydronaphthalen-2- | 4C(CC(C)C(C4)(C)C(C)═ | wood, ketonic, phenolic | |
| | | | yl)ethanone; 1-(2,3,8,8- | O)═CCC3.CC5(C)CCCC6═ | | |
| | | | tetramethyl-1,3,5,6,7,8~{a}- | CC(C)C(C(C)═O)(C)CC56 | | |
| | | | hexahydronaphthalen-2- | | | |
| | | | yl)ethanone; 1-(2,3,8,8- | | | |
| | | | tetramethyl-1,3,4,5,6,7- | | | |
| | | | hexahydronaphthalen-2- | | | |
| | | | yl)ethanone | | | |
| 3. | Z-95 | 113667-25-7 | (4~{a}~{S},8~{a}~{R})- | O═C(CC[C@@]12[H])C[ | woody, amber, | 82% |
| | | | 5,5,8~{a}-trimethyl- | C@@]1(CCCC2(C)C)C | camphor | |
| | | | 3,4,4~{a},6,7,8-hexahydro- | | | |
| | | | 1~{H}-naphthalen-2-one | | | |
| 4. | Trimofix | 144020-22-4 | 1-(4,8-dimethyl-12- | CC1═CCCC(C)═C(C(C)═ | ambergris, woody, | 74% |
| | | | methylidenecyclododeca-4,8- | O)CCC(C)═CCC1.CC2═C | cedar, dry, tobacco, | |
| | | | dien-1-yl)ethanone; 1-(2,6,10- | CC═C(C)C(C(C)═O)CCC( | musk | |
| | | | trimethylcyclododeca-1,5,9- | C)═CCC2.CC(CCC═C(C) | | |
| | | | trien-1-yl)ethanone; 1-(2,6,10- | CCC3C(C)═O)═CCCC3═C | | |
| | | | trimethylcyclododeca-2,5,9- | | | |
| | | | trien-1-yl)ethanone | | | |
| 5. | Methyl | 15789-90-9 | (+−)-(e)-3-methyl-4-(2,6,6- | CC(═O)/C(═C/C1C(═CCC | violet, sweet, orris, | 65% |
| | ionone | | trimethyl-2-cyclohexen-1-yl)- | C1(C)C)C)C | powdery, floral, | |
| | gamma | | 3-buten-2-one | | woody, fruity, tea | |

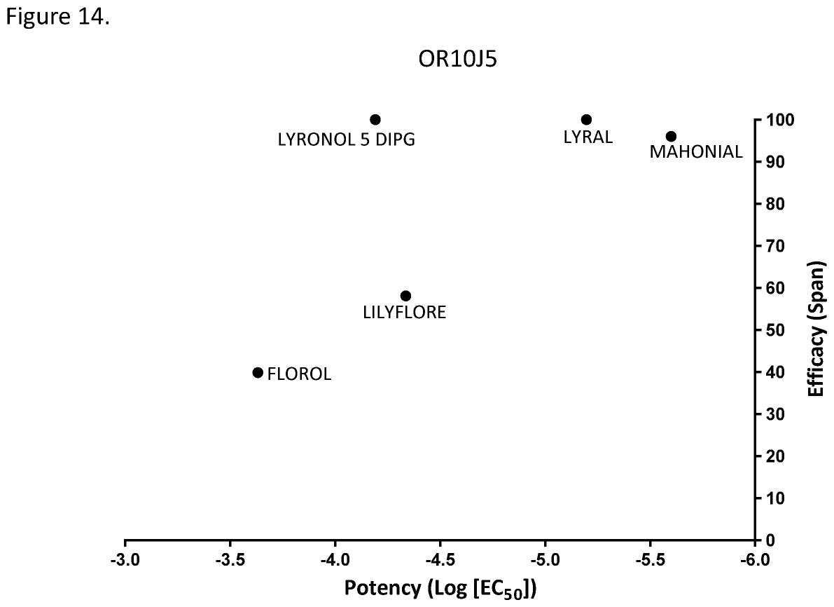

OR10J5, SEQ ID NO. 27, was activated by compounds generating a muguet tonality (FIG. 14, Table 10).

TABLE 10
Compounds activating OR10J5 share the olfactory muguet tonality.

| | Compound | CAS | IUPAC | | Organoleptic | OR10J5 |
|---|---|---|---|---|---|---|
| | Name | No | Name | Smiles | Descriptors | % Efficacy |
| 1. | Lyral | 031906-04-4 | 3-(4-hydroxy-4- | O═CC1CC(CCCC(C)(C)O)═ | lily of the valley, | 100% |
| | | | methylpentyl)cyclohex-3- | CCC1.OC(C)(C)CCCC2═ | aldehydic, watery | (full |
| | | | ene-1-carbaldehyde; 4-(4- | CCC(C═O)CC2 | | agonist) |
| | | | hydroxy-4- | | | |
| | | | methylpentyl)cyclohex-3- | | | |
| | | | ene-1-carbaldehyde | | | |
| 2. | Lyronol | 067634-12-2 | methyl 2-[[3-(4-hydroxy-4- | O═C(OC)C1═CC═CC═C1 | floral, aldehydic, | 100% |
| | 50 dipg | | methylpentyl)cyclohex-3-en-1- | N/C═C2CCC(CCCC(C)(O) | lily of the valley, | (full |
| | | | ylidene]methylamino]benzoate; | C)═CC/2.O═C(OC)C3═CC═ | orange blossom | agonist) |
| | | | methyl 2-[[4-(4-hydroxy- | CC═C3N/C═C4CC═C(C | | |
| | | | 4-methylpentyl)cyclohex-3 - | CCC(C)(O)C)CC/4.O═C(O | | |
| | | | en-1- | C)C5═CC═CC═C5N/C═C6 | | |
| | | | ylidene]methylamino]benzoate | CCC═C(CCCC(C)(O)C)C/ | | |
| | | | | 6.O═C(OC)C7═CC═CC═C | | |
| | | | | 7N/C═C8CC(CCCC(O)(C) | | |
| | | | | C)═CCC/8 | | |
| 3. | Mahonial | 926-50-1 | 9-hydroxy-5,9-dimethyldec- | OC(C)(C)CCC/C(C)═C\CC | Floral, muguet | 96% |
| | | | 4-enal | C═O.OC(C)(C)CCC/C(C)═C/ | | |
| | | | | CCC═O | | |
| 4. | Lilyflore | 285977-85-7 | (2,5-dimethyl-1,3- | CC1═CC(CC(C)(CO)C2)═ | aldehydic, lilial, | 58% |
| | | | dihydroinden-2-yl)methanol | C2C═C1 | hydroxycitronellal, | |
| | | | | | muguet | |
| 5. | Florol | 65418-69-1 | 4-methyl-2-(2- | CC1(CC(CC(C)C)OCC1)O | lily of the valley, | 40% |
| | | | methylpropyl)oxan-4-ol | | watery | |

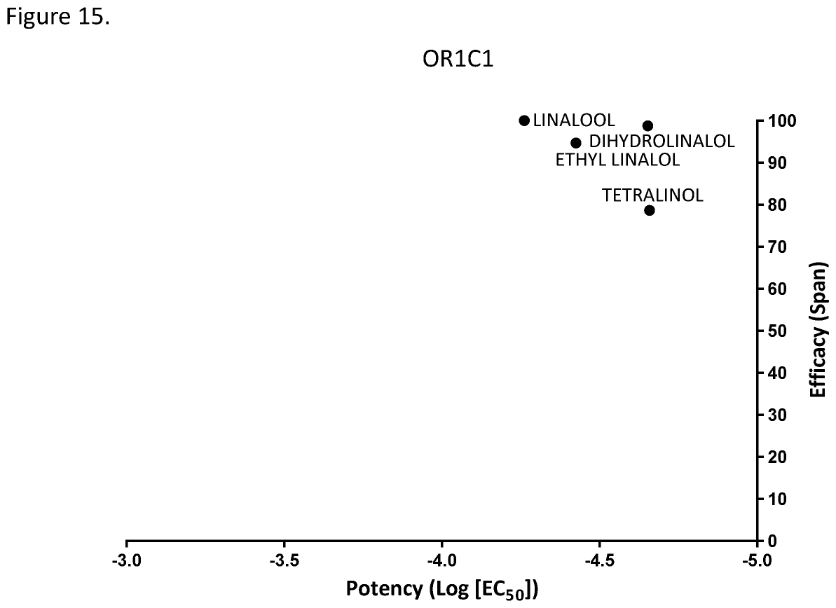

OR1C1, SEQ ID NO. 29, was activated by compounds generating a linalic tonality (FIG. 15, Table 11).

TABLE 11
Compounds activating OR1C1 share the olfactory linalic tonality.

| | Compound | CAS | IUPAC | | Organoleptic | OR1C1 |
|---|---|---|---|---|---|---|
| | Name | No | Name | Smiles | Descriptors | % Efficacy |
| 1. | Dihydrolina | 018479-49-7 | 3,7-dimethyloct-1-en-3-ol | CC(C)CCCC(C═C)(O)C | nicely floral, dimetol, | 95% |
| | lol | | | | linalool, fresh | |
| 2. | Linalool | 000078-70-6 | 3,7-dimethylocta-1,6-dien-3-ol | CC(C═C)(O)CCC═C(C)C | linalool, green, | 100% |
| | | | | | terpenic, | (full |
| | | | | | camphoraceous, | agonist) |
| | | | | | pine, lime, bornyl | |
| 3. | Ethyl | 92590-71-1 | 3,7-dimethylnona-1,6-dien-3-ol | CC(C═C)(O)CC/C═C(CC)\ | floral, linalool, oily, | 99% |
| | Linalool | | | C.CC(C═C)(O)CC/C═C(C)\ | volatile | |
| | | | | CC | | |
| 4. | Tetralinol | 000078-69-3 | 3,7-dimethyloctan-3-ol | CC(CC)(O)CCCC(C)C | linalool | 79% |

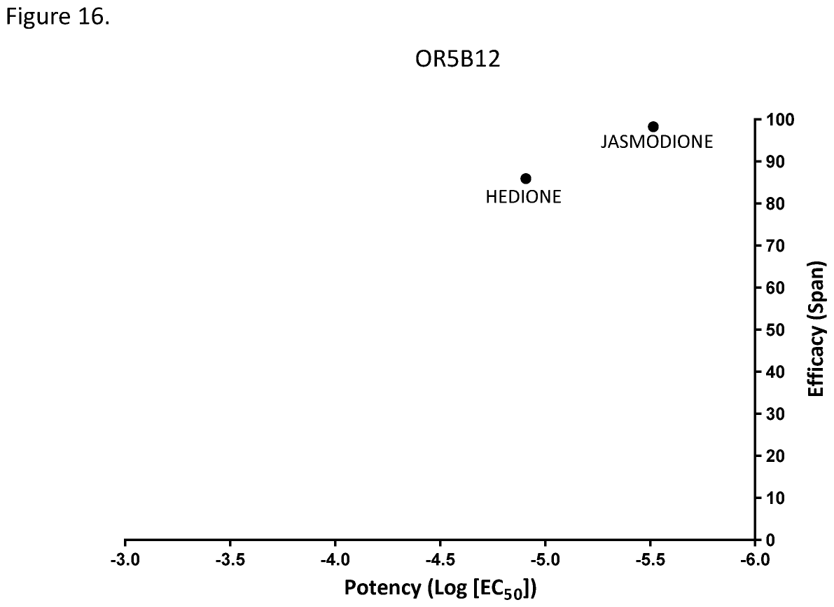

OR5B12, SEQ ID NO. 31, was activated by compounds generating a jasmine tonality (FIG. 16, Table 12).

TABLE 12
Compounds activating OR5B12 share the olfactory jasmine tonality.

| | Compound | CAS | IUPAC | | Organoleptic | OR5B12 |
|---|---|---|---|---|---|---|
| | Name | No | Name | Smiles | Descriptors | % Efficacy |
| 1. | Jasmodione | 39647-11-5 | methyl 2-[(1~{S},2~{S})-3- | O═C1CC[C@@H](CC(OC)═ | floral, jasmine, | 98% |
| | | | oxo-2- | O)[C@H]1CCCCC.O═C2 | estery | |
| | | | pentylcyclopentyl]acetate; | [C@@H](CCCCC)[C@H | | |
| | | | methyl 2-[(1~{R},2~{S})-3- | ](CC(OC)═O)CC2 | | |
| | | | oxo-2- | | | |
| | | | pentylcy clopentyl]acetate | | | |
| 2. | Hedione | 24851-98-7 | methyl 2-[(1~{R},2~{S})-3- | O═C1[C@@H](CCCCC)[ | floral, jasmine, | 86% |
| | | | oxo-2- | C@H](CC(OC)═O)CC1 | rancid, weak | |
| | | | pentylcy clopentyl]acetate | | | |

### Example 4: Use of Odorant Receptor Activity to Analyse and Generate Complex Mixtures

Odorant receptor activity as a whole is used to determine the fragrance of a composition containing multiple volatile molecules. The composition's activity on an ensemble of ORs reflects the final activity achieved by the composition on each OR, taking into account OR modulation (e.g. inhibition and enhancement), that may occur when ORs are contacted by multiple compounds. The resulting activity profile is used to describe the overall tonalities of the composition using the links established between OR activity and perceived tonality. Alternatively, the activity profile required for a target smell can be inferred and used as a signature activity to reproduce in order to recreate the target smell in distinct conditions. Similarly, such signature activity profiles can be used as a quality evaluation or quality standard tests.

The composition activity is also used as a signature to guide the creation of a new composition that recapitulates the activity of the original composition. Such a new composition is obtained by modifying the initial composition while retaining the original ensemble of activated ORs. Such modification may include removing an irrelevant compound, replacing a compound with a preferred one, replacing one compound with multiple preferred compounds, replacing multiple compounds with one compound that plays the same role as the multitude of compounds being replaced, or replacing multiple compounds with multiple distinct compounds. A new composition with similar or identical activity as the target composition is similar in odorant characteristics.

Finally, a composition is created anew by inferring the compounds required to obtain an intended fragrance. For example, a composition is created with a set of desired tonalities. Alternatively, a composition can be engineered to remove one or more undesired tonalities such as malodors, for example. A composition can also be created employing both strategies concomitantly.

### Example 5: OR Activity for Complex Mixtures can be Modelled and Lead to Perceivable Tonality Prediction

A perfumery accord (accord A) contained ingredients activating odorant receptors OR8B3 (contributing a hay tonality), OR8D1 (contributing a celery tonality) and OR5AU1 (contributing a lactonic coconut tonality) along with three accords in which small modifications were introduced (accords B-D). See Table 12.

TABLE 12

| | Composition Ratio |
|---|---|
| Accord A | |
| NONALACTONE GAMMA | 25.92 |
| CYCLOTONE | 1.31 |
| Other Ingredients | 8.65 |
| DIPROPYLENE GLYCOL | 64.12 |
| Accord B | |
| NONALACTONE GAMMA | 25.92 |
| CYCLOTONE | — |
| Other Ingredients | 8.65 |
| MAPLE FURANONE | 0.0017 |
| DIPROPYLENE GLYCOL | 65.43 |
| Accord C | |
| NONALACTONE GAMMA | — |
| CYCLOTONE | 1.31 |
| Other Ingredients | 8.65 |
| COUMARIN | 3.95 |
| DIPROPYLENE GLYCOL | 86.09 |
| Accord D | |
| NONALACTONE GAMMA | — |
| CYCLOTONE | 1.31 |
| Other Ingredients | 8.65 |
| COUMARIN | 3.95 |
| TUBEROLIDE | 2.043 |
| DIPROPYLENE GLYCOL | 84.09 |

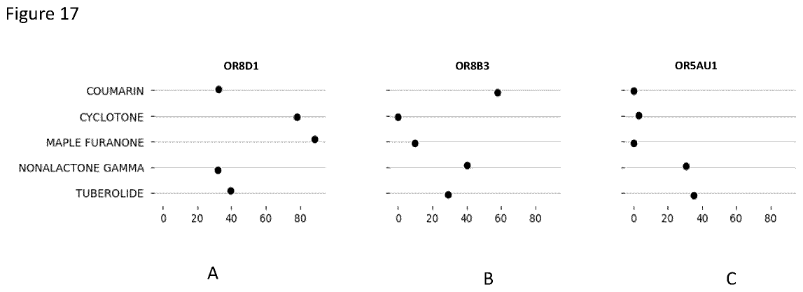

Ingredients that do not activate any of the three target ORs were aggregated for simplification and described as “other ingredients”. Receptor screening data (as described in Example 1) was used for each OR to identify alternative activators that can be used to replace an ingredient present in an original formula. This approach allowed the use of favored ingredients to be prioritized for the optimization of multiple factors including decreasing cost, increasing biodegradability, or using compounds with environmentally friendly properties. The overall activity of the accords on each receptor can be calculated with a competitive binding model by considering the ingredient ratio in the mixture as shown in Table 12, as well as their individual activity levels for each receptor as shown in FIG. 17. Activity levels were normalized to the maximal cellular response to Forskolin, a known pharmacological transduction cascade activator and serves as anchor point for data comparison purposes. The accord activity predicted by the model were compared to cell-based data generated with the same accords (cell-based assay as described in Example 2). Congruent activity levels were observed between model and in vitro data, validating the model. The resulting activity predictions between original and modified accords were used to make sensory predictions based on OR activity changes (i.e. more or less active) and inferring the corresponding tonality change (i.e. more or less intense). The sensory predictions were validated by perfumery expert sensory panel asked to perform a blind evaluation of the three tonalities of interest: hay, celery and lactonic coconut, for each accord.

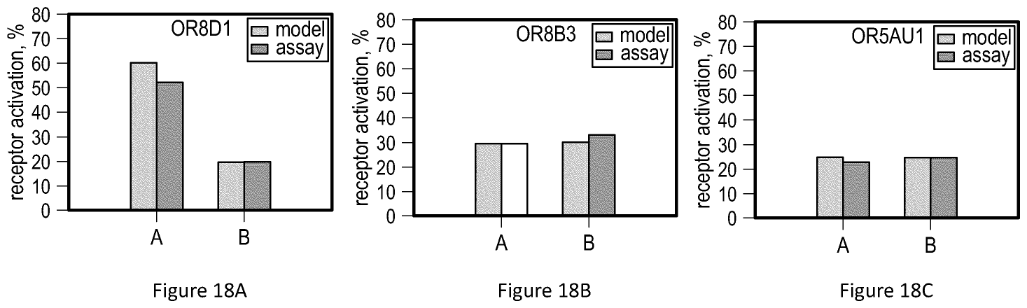

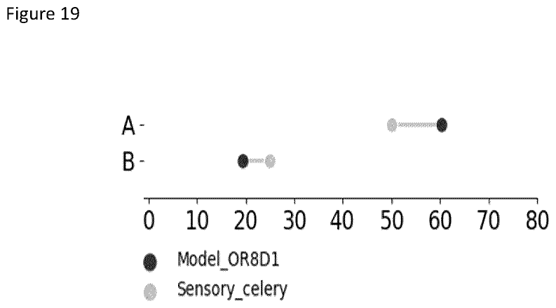

The case of a composition in which cyclotone was removed and replaced with maple furanone with respect to OR8D1 activity is shown by comparing accord A and B. The target activity level was set to be lower for OR8D1 versus original. FIG. 18 shows the predicted activity level from the model in comparison to the in vitro validation for the receptor, normalized to 100% Forskolin response. A corresponding reduction in the perception of the celery tonality was predicted which was confirmed by the expert panelist evaluation as summarized in FIG. 19.

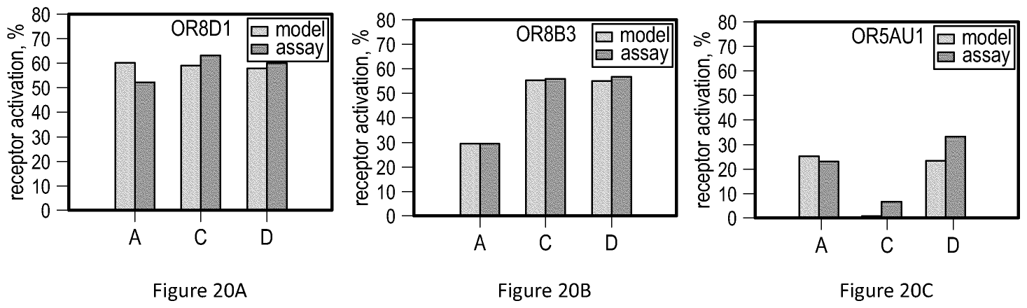

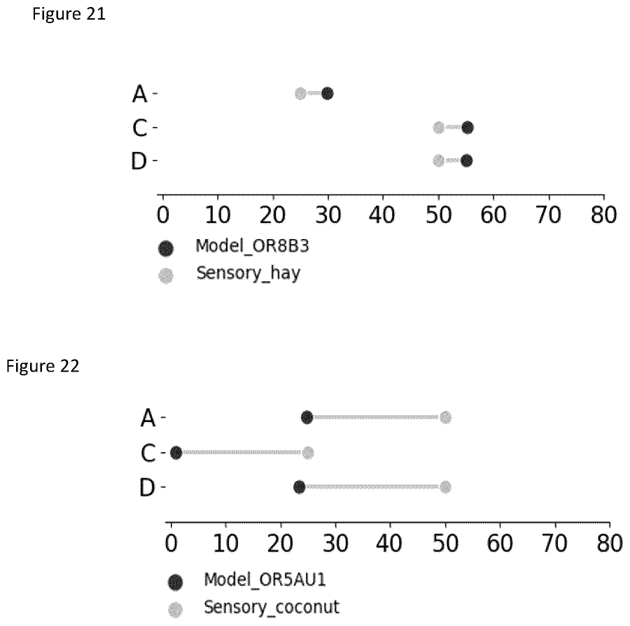

The case of a composition in which nonalactone was removed and replaced with coumarin with respect to OR8B3 and OR5AU1 activity is shown by comparing accords A and C. In this case, the target activity level was set to be higher for OR8B3 and null for OR5AU1 versus original. FIG. 20 shows the predicted activity level from the model in comparison to the in vitro validation for both receptors, normalized to 100% Forskolin response. A corresponding increase in the perception of the hay and loss of the lactonic coconut tonalities were predicted which was confirmed by the expert panelist evaluation as summarized in FIG. 21.

The case of a composition in which nonalactone was removed, replaced with coumarin, and tuberolide was added with respect to OR8B3 and OR5AU1 activity is shown by comparing accords A, C and D. In this case, the target activity level was set to be unchanged for OR8B3 versus accord C and restored for OR5AU1 versus original. FIG. 20 shows the predicted activity level from the model in comparison to the in vitro validation for both receptors, normalized to 100% Forskolin response. A corresponding recovery of the perception of the coconut tonality was predicted which was confirmed by the expert panelist evaluation as summarized in FIG. 22.

These perfumery accord alteration examples captured the system's ability to model receptor activity for complex mixtures before and after removal and replacements of compounds using solely single compound input data (as obtained in Example 1-3). The ability of the system to predict sensory outcome based on receptor activity calculation was also evidenced for maintaining the perception of a specific desired tonality with distinct compounds; adjusting the replacement compound concentration or ratio in the mixture to decrease or increase a desired tonality; and for adding a compound to rebalance a composition by restoring a desired tonality lost during alterations.

The contents of all patents, publications, and published patent applications cited herein are hereby incorporated by reference in their entirety.

1. A method for associating an at least one olfactory tonality to an olfactory receptor comprising:
(a) providing an olfactory receptor;
(b) contacting the olfactory receptor and a compound having at least one known tonality;
(c) determining whether the compound activates the olfactory receptor;
(d) repeating steps (b) and (c) with a compound having at least one known tonality, wherein the compound of step (d) is different than the compound of the preceding iterations of steps (b) and (c);
(e) categorizing as a subset, the compounds from steps (b) through (d) that activate the olfactory receptor;
(f) identifying an at least one tonality common to the subset of compounds; and
(g) assigning the identified at least one tonality to the olfactory receptor.

2. A method for screening at least one compound having a particular tonality comprising:
(a) providing an olfactory receptor having the identified at least one tonality of claim 1;
(b) contacting the olfactory receptor with an at least one compound;
(c) determining whether the at least one compound activates the olfactory receptor;
(d) associating the at least one compound to the identified at least one tonality if the at least one compound activates the olfactory receptor.

3. The method of claim 2, wherein a plurality of olfactory receptors, each having a different identified at least one tonality, is provided in step (a) and a plurality of identified tonalities is associated in step (d) to the compound or combination of compounds that activate the plurality of olfactory receptors according to steps (b) and (c).

4. A method for screening at least one compound for an earthy tonality comprising:
(a) providing a polypeptide comprising an amino acid sequence having at least 90% sequence identity to SEQ ID NO:11;
(b) contacting the polypeptide with the at least one compound;
(c) determining whether the at least one compound activates the polypeptide; and
(d) associating an earthy tonality to the at least one compound if the at least one compound activates the polypeptide;
wherein the polypeptide is an olfactory receptor.

5. A method for screening at least one compound for a coumarinic tonality comprising:
(a) providing a polypeptide comprising an amino acid sequence having at least 90% sequence identity to SEQ ID NO:13;
(b) contacting the polypeptide with the at least one compound;
(c) determining whether the at least one compound activates the polypeptide; and
(d) associating a coumarinic tonality to the at least one compound if the at least one compound activates the polypeptide;
wherein the polypeptide is an olfactory receptor.

6. A method for screening at least one compound for a lactonic coconut tonality comprising:
(a) providing a polypeptide comprising an amino acid sequence having at least 90% sequence identity to SEQ ID NO:15;
(b) contacting the polypeptide with the at least one compound;
(c) determining whether the at least one compound activates the polypeptide; and
(d) associating an lactonic coconut tonality to the at least one compound if the at least one compound activates the polypeptide;
wherein the polypeptide is an olfactory receptor.

7. A method for screening at least one compound for a fenugreek tonality comprising:
(a) providing a polypeptide comprising an amino acid sequence having at least 90% sequence identity to SEQ ID NO:17;
(b) contacting the polypeptide with the at least one compound;
(c) determining whether the at least one compound activates the polypeptide; and
(d) associating a fenugreek tonality to the at least one compound if the at least one compound activates the polypeptide;
wherein the polypeptide is an olfactory receptor.

8. A method for screening at least one compound for a powdery musk tonality comprising:
(a) providing a polypeptide comprising an amino acid sequence having at least 90% sequence identity to SEQ ID NO:19;
(b) contacting the polypeptide with the at least one compound;
(c) determining whether the at least one compound activates the polypeptide; and
(d) associating a powdery musk tonality to the at least one compound if the at least one compound activates the polypeptide;
wherein the polypeptide is an olfactory receptor.

9. A method for screening at least one compound for an animalic musk tonality comprising:
(a) providing a polypeptide comprising an amino acid sequence having at least 90% sequence identity to SEQ ID NO:21;
(b) contacting the polypeptide with the at least one compound;
(c) determining whether the at least one compound activates the polypeptide; and
(d) associating an animalic musk tonality to the at least one compound if the at least one compound activates the polypeptide;
wherein the polypeptide is an olfactory receptor.

10. A method for screening at least one compound for a violet tonality comprising:
(a) providing a polypeptide comprising an amino acid sequence having at least 90% sequence identity to SEQ ID NO:23;
(b) contacting the polypeptide with the at least one compound;
(c) determining whether the at least one compound activates the polypeptide; and
(d) associating a violet tonality to the at least one compound if the at least one compound activates the polypeptide;
wherein the polypeptide is an olfactory receptor.

11. A method for screening at least one compound for a blonde wood comprising:
(a) providing a polypeptide comprising an amino acid sequence having at least 90% sequence identity to SEQ ID NO:25;
(b) contacting the polypeptide with the at least one compound;
(c) determining whether the at least one compound activates the polypeptide; and
(d) associating a blonde wood tonality to the at least one compound if the at least one compound activates the polypeptide;
wherein the polypeptide is an olfactory receptor.

12. A method for screening at least one compound for a muguet tonality comprising:
(a) providing a polypeptide comprising an amino acid sequence having at least 90% sequence identity to SEQ ID NO:27;
(b) contacting the polypeptide with the at least one compound;
(c) determining whether the at least one compound activates the polypeptide; and
(d) associating a muguet tonality to the at least one compound if the at least one compound activates the polypeptide;
wherein the polypeptide is an olfactory receptor.

13. A method for screening at least one compound for a linalic tonality comprising:
(a) providing a polypeptide comprising an amino acid sequence having at least 90% sequence identity to SEQ ID NO:29;
(b) contacting the polypeptide with the at least one compound;
(c) determining whether the compound or combination of compounds activates the polypeptide; and
(d) associating a linalic tonality to the at least one compound if the at least one compound activates the polypeptide;
wherein the polypeptide is an olfactory receptor.

14. A method for screening at least one compound for a jasmine tonality comprising:
(a) providing a polypeptide comprising an amino acid sequence having at least 90% sequence identity to SEQ ID NO:31;
(b) contacting the polypeptide with the at least one compound;
(c) determining whether the compound or combination of compounds activates the polypeptide; and
(d) associating a jasmine tonality to the at least one compound if the at least one compound activates the polypeptide;
wherein the polypeptide is an olfactory receptor.

15. At least one compound identified by the method according to claim 2.

16. At least one compound identified by the method according to claim 4.

17. At least one compound identified by the method according to claim 5.

18. At least one compound identified by the method according to claim 6.

19. At least one compound identified by the method according to claim 7.

20. At least one compound identified by the method according to claim 8.
